(19) United States
(12) Patent Application Publication
(10) Pub. No.: US 2025/0364085 A1
(43) Pub. Date: Nov. 27, 2025

(54) METHOD FOR IDENTIFYING POST-TRANSLATIONAL MODIFICATIONS IN CROSS-LINKING MASS SPECTROMETRY DATA

(71) Applicant: The Hong Kong University of Science and Technology, Kowloon (HK)

(72) Inventors: Weichuan YU, Kowloon (HK); Ning LI, Kowloon (HK); Chen ZHOU, Kowloon (HK); Shengzhi LAI, Kowloon (HK)

(21) Appl. No.: 19/172,944

(22) Filed: Apr. 8, 2025

Related U.S. Application Data
(60) Provisional application No. 63/651,411, filed on May 24, 2024.

(51) Int. Cl. G16B 40/10; G16B 30/00; H01J 49/00

(52) U.S. Cl. CPC G16B 40/10; G16B 30/00; H01J 49/0036

(57) ABSTRACT

A system, for identifying post-translational modification in cross-linking mass spectrometry data, can comprise at least one processor, and at least one memory that stores executable instructions that, when executed by the at least one processor, facilitates performance of operations, comprising generating a peptide sequence tag graph based on a dataset of cross-linking spectral (XL-MS) data defining a real peptide set of one or more peptides and on a peptide database comprising information defining known peptides, based on a fuzzy string matching process applied to the peptide sequence tag graph, identifying a candidate peptide set corresponding to the real peptide set, identifying a post-translational modification (PTM) within the candidate peptide set, and scoring the PTM based on an aggregation of additional identifications of the PTM, wherein the scoring results in a PTM score assigned to the PTM that defines a probability of the PTM being comprised by the real peptide set.

### CROSS REFERENCE TO RELATED APPLICATION

This is a nonprovisional patent application claiming priority under 35 U.S.C. § 119 to U.S. Provisional Patent Application No. 63/651,411, filed on May 24, 2024, and entitled “METHOD FOR IDENTIFYING POST-TRANSLATIONAL MODIFICATIONS IN CROSS-LINKING MASS SPECTROMETRY DATA,” the entirety of which priority application is hereby incorporated by reference herein.

### BACKGROUND

Cross-linking mass spectrometry (XL-MS) is a technique for studying protein-protein interactions (PPIs) and protein structural conformations in a high throughput manner.

### SUMMARY

The following presents a simplified summary of the disclosed subject matter to provide a basic understanding of one or more of the various embodiments described herein. This summary is not an extensive overview of the various embodiments. It is intended neither to identify key or critical elements of the various embodiments nor to delineate the scope of the various embodiments. Its sole purpose is to present one or more concepts of the disclosure in a streamlined form as a prelude to the more detailed description that is presented later.

Described herein are one or more frameworks directed to identifying the aforementioned post-translational modifications within cross-linking mass spectrometry data. These identifications and analysis data associated therewith can be employed as determined, and/or can be input into another XL-MS search for use as initially tuned data.

An example system can comprise at least one processor, and at least one memory that stores executable instructions that, when executed by the at least one processor, facilitates performance of operations, comprising generating a peptide sequence tag graph based on a dataset of cross-linking spectral (XL-MS) data defining a real peptide set of one or more peptides and based on a peptide database comprising information defining known peptides, based on a fuzzy string matching process applied to the peptide sequence tag graph, identifying a candidate peptide set corresponding to the real peptide set, identifying a post-translational modification (PTM) within the candidate peptide set, and scoring the PTM based on an aggregation of additional identifications of the PTM, wherein the scoring results in a PTM score assigned to the PTM that defines a probability of the PTM being comprised by the real peptide set.

An example method can comprise generating, by a system comprising at least one processor, a peptide sequence tag graph based on a dataset of cross-linking spectral (XL-MS) data defining a real peptide set of one or more peptides and a peptides database comprising information defining peptides, the generating comprising identifying mass differences between pairs of peaks, of the XL-MS data along the mass/charge (m/z) axis, as corresponding to masses of known amino acids, generating tags having respective lengths of one amino acid, and generating the peptide sequence tag graph comprising the tags. The method can further comprise identifying a candidate peptide set from the peptide sequence tag graph and corresponding to the real peptide set, identifying a post-translational modification (PTM) within the candidate peptide set, and scoring the PTM resulting in a PTM score assigned to the PTM that defines a probability of the PTM being comprised by the real peptide set.

An example non-transitory machine-readable medium can comprise executable instructions that, when executed by at least one processor facilitate performance of operations, comprising generating a peptide sequence tag graph based on a dataset of cross-linking spectral (XL-MS) data defining a real peptide set of one or more peptides and a protein database comprising information defining proteins and evaluating amino acid tags of the peptide sequence tag graph, comprising exploring possible paths from starting nodes to end nodes of a system of nodes of the peptide sequence tag graph, ranking each path based on a combination of length and sum of weighted intensity thereof, and employing top ranking paths, from the ranking, and that each comprise at least four amino acids, as input to a fuzzy string matching process. The operations further can comprise, based on the fuzzy string matching process, identifying a candidate peptide set corresponding to the real peptide set, identifying a post-translational modification (PTM) within the candidate peptide set, and scoring the PTM based on an aggregation of additional identifications of the PTM, wherein the scoring results in a PTM score assigned to the PTM that defines a probability of the PTM being comprised by the real peptide set.

An example benefit of one or more of the above-indicated example embodiments can be an ability to provide an input set of data, comprising probable PTMs and/or peptide matches, to an XL-MS search engine, allowing for a more reliable and/or efficient analysis by the XL-MS search engine. That is, the XL-MS technique is a data-heavy and time-intensive technique that can be sped up using a combination of the one or more embodiments described herein and an existing XL-MS search engine using output of the one or more embodiments described herein. Put another way, the screening capabilities provided by the one or more embodiments described herein can extract PTM information from data, and enhance the performance of existing XL-MS search engines. Indeed, using the one or more embodiments described herein, PTM information can be identified that is otherwise not identified using only an existing XL-MS search engine.

Another example benefit of one or more of the above-indicated example embodiments can be the versatility of use of the one or more embodiments described herein. That is, the method of searching PTMs in cross-linking mass spectrometry data (SeaPIC) described herein can be employed with different spectrum types, cross-linker (e.g., cross-linking reagent) types and/or task types (e.g., cross-linked peptides vs. linear peptides). For example, SeaPIC can be employed with various types of spectra, including, but not limited to, collision-induced dissociation (CID) spectra, high-energy collisional dissociation (HCD) spectra, and electron-transfer dissociation (ETD) spectra, and is capable of addressing both non-cleavable and cleavable cross-linking scenarios.

Still another example benefit of one or more of the above-indicated example embodiments can be an ability to generate a database comprising the data generated by the one or more embodiments described herein. This can comprise, but is not limited to, spectrum peptide paths, candidate peptides, generated sequence tags and/or corresponding PTMs.

### BRIEF DESCRIPTION OF THE DRAWINGS

The technology described herein is illustrated by way of example and not limited to the accompanying figures in which like reference numerals indicate similar elements.

FIG. 1 illustrates a block diagram of an example, non-limiting, post-translational modification (PTM) identifying system, in accordance with one or more example embodiments and/or implementations described herein.

FIG. 2 illustrates example aspects of sequence tag graph generation and depth first search, of a workflow for identifying a PTM using the non-limiting system of FIG. 1, in accordance with one or more example embodiments and/or implementations described herein.

FIG. 3 illustrates an example aspect of fuzzy string matching, of a workflow for identifying a PTM using the non-limiting system of FIG. 1, in accordance with one or more example embodiments and/or implementations described herein.

FIG. 4 illustrates example aspects of peptide filtering, fast PTM search and score regularization, of a workflow for identifying a PTM using the non-limiting system of FIG. 1, in accordance with one or more example embodiments and/or implementations described herein.

FIG. 5 illustrates an example algorithm for a fast PTM search, as can be performed by the non-limiting system of FIG. 1, in accordance with one or more example embodiments and/or implementations described herein.

FIG. 6 illustrates an example case of experimental PTM scoring, as can be performed by the non-limiting system of FIG. 1, in accordance with one or more example embodiments and/or implementations described herein.

FIG. 7 illustrates an example graph demonstrating correlation of a weight term, of a scoring function employed for PTM scoring, to quantity of PTMs, in accordance with one or more example embodiments and/or implementations described herein.

FIG. 8 illustrates example aspects of result normalization, of a workflow for identifying a PTM using the non-limiting system of FIG. 1, in accordance with one or more example embodiments and/or implementations described herein.

FIG. 9 illustrates example aspects of export of PTM information, of a workflow for identifying a PTM using the non-limiting system of FIG. 1, in accordance with one or more example embodiments and/or implementations described herein.

FIG. 10 illustrates example simulation results that can be generated by the non-limiting system of FIG. 1, in accordance with one or more example embodiments and/or implementations described herein.

FIG. 11 illustrates example synthetic results that can be generated by the non-limiting system of FIG. 1, in accordance with one or more example embodiments and/or implementations described herein.

FIG. 12 illustrates example synthetic results that can be generated by the non-limiting system of FIG. 1, in accordance with one or more example embodiments and/or implementations described herein.

FIG. 13 illustrates example real experimental results that can be generated by the non-limiting system of FIG. 1, in accordance with one or more example embodiments and/or implementations described herein.

FIG. 14 illustrates example real experimental results that can be generated by the non-limiting system of FIG. 1, in accordance with one or more example embodiments and/or implementations described herein.

FIG. 15 illustrates example real experimental results that can be generated by the non-limiting system of FIG. 1, in accordance with one or more example embodiments and/or implementations described herein.

FIG. 16 illustrates an example process flow diagram of example processes that can be performed by the non-limiting system of FIG. 1, in accordance with one or more example embodiments and/or implementations described herein.

FIG. 17 illustrates a continuation of the example process flow diagram of FIG. 16, in accordance with one or more example embodiments and/or implementations described herein.

FIG. 18 illustrates a continuation of the example process flow diagram of FIG. 17, in accordance with one or more example embodiments and/or implementations described herein.

FIG. 19 illustrates a continuation of the example process flow diagram of FIG. 18, in accordance with one or more example embodiments and/or implementations described herein.

FIG. 20 illustrates a block diagram of an example operating environment into which embodiments of the subject matter described herein can be incorporated.

FIG. 21 illustrates a schematic block diagram of an example computing environment with which the subject matter described herein can interact and/or be implemented at least in part.

### DETAILED DESCRIPTION

### Overview

The technology described herein is generally directed towards, for example, devices, systems, methods and/or non-transitory mediums for identification of post-translational modifications and generation of corresponding PTM scores based on input cross-linking mass spectrometry (XL-MS) data. For example, one or more embodiments described herein can provide for searching and identifying of PTMs in cross-linking mass spectrometry data, also herein referred to as a SeaPIC (searching PTMs in cross-linking mass spectrometry data) technique and/or method.

Mass spectrometry, generally, refers to the measuring of a mass-to-charge (or mass/charge) ratio of one or more molecules of a sample, such as of a precursor or molecules broken down from a precursor.

Compared to other existing MS techniques, XL-MS uses a cross-linker to react with protein complexes before acquiring MS data. Cross-linkers are chemical compounds typically consisting of two reaction groups. During experiments, these reaction groups form covalent bonds to specific amino acids in proteins. In a cellular context, when two proteins interact, indicating their spatial proximity, a cross-linker reacts with them, capturing this PPI information. Cross-linkers can also react with amino acids in the same protein but at different spatial positions, enabling the inference of topological information regarding proteins' structural conformations. After cross-linkers react with target protein samples, XL-MS follows the traditional MS procedure to generate the first-stage MS (MS1) spectra and second-stage MS (MS2) spectra.

The analysis of MS2 spectra in XL-MS can present a non-trivial computational challenge. Computing cross-link spectrum matches (CSMs) can involve considering the combination of any two peptides in a protein sequence database, resulting in a quadratic searching space problem. Existing frameworks have designed cleavable cross-linkers and introduced third-stage MS (MS3) spectra, simplifying the problem to a linear peptide task from a wet lab aspect. Tool developers have designed advanced two-step searching methods and exhaustive searching algorithms that can also handle the task with linear time complexity from the dry lab perspective.

However, these advancements only address the fundamental computational challenge in XL-MS data interpretation without considering the occurrence of post-translational modifications (PTMs).

Post-translational modifications (PTMs), in molecular biology, are covalent processes of changing proteins following protein biosynthesis. These biochemical changes can occur to a protein after the protein has been synthesized and translated from mRNA. Types of PTMs can comprise, but are not limited to phosphorylation, methylation, acetylation, glycosylation, ubiquitination, small ubiquitin-like modifier-ylation (SUMOylation), prenylation, hydroxylation, proteolysis and/or acylation.

A PTM can alter one or more of a structure, function and/or localization of one or more proteins, thereby playing an impactful role in various cellular processes. Indeed, PTMs can play an impactful role in regulating biological processes and enabling protein-protein interactions. PTMs also can impact protein folding, stability and/or conformational changes.

Therefore, using XL-MS data generally, the identification of, and subsequent analysis of, PTMs within cross-linked peptides can shed light on various biological processes, if such PTMs can be identified. Unfortunately, existing frameworks fail to provide identification of PTMs, misidentify PTMs and/or identify less than all PTMs. This can be because studying PTMs when using an XL-MS technique involves more than merely identifying peptide sequences. As such, although use of PTMs can offer valuable insights, their identification remains a complex, problem-fraught and/or relatively unexplored technical area.

To make up for one or more deficiencies of existing frameworks, generally, the one or more embodiments described herein can enable scientists to uncover PTMs in XL-MS data regardless of the type of spectra of the XL-MS data. For example, SeaPIC can be employed with various types of spectra, including, but not limited to, collision-induced dissociation (CID) spectra, high-energy collisional dissociation (HCD) spectra, and electron-transfer dissociation (ETD) spectra, and is capable of addressing both non-cleavable and cleavable cross-linking scenarios. Moreover, SeaPIC can be used in combination with existing XL-MS search engines.

To provide these benefits, SeaPIC performed by the one or more embodiments described herein, can serve as a screening method, utilizing generated tag information, describing the XL-MS data, to determine a partial sequence of cross-linked peptides, corresponding to the XL-MS data, and to solve for (e.g., identify) potential PTMs in the sequence. This can be accomplished without the need to decipher the complete pairs of cross-linked peptides of the partial sequence. The workflow of SeaPIC, as can be performed by the one or more embodiments described herein, can encompass a plurality of steps including, but not limited to, sequence tag graph construction, a depth first search, fuzzy string matching, peptide filtering, fast PTM search, score regularization, result normalization, and/or the export of PTM information, such as to an existing XL-MS search engine. By employing SeaPIC's screening capabilities, researchers can extract PTM information from XL-MS data and/or can enhance the performance of existing XL-MS search engines by using output of the one or more embodiments described herein as input to an existing XL-MS search engine.

It is noted that existing XL-MS search engines do not provide the filtering and/or screening that can be provided by the one or more embodiments described herein, including failing to provide the aforementioned workflow aspects, whether separately or in any combination thereof.

### Terminology

As used herein, the terms “cost” or “expense” can refer to power, memory, and/or processing power.

As used herein, the term “data” can comprise “metadata.”

Reference throughout this specification to “embodiment,” “one embodiment,” “an embodiment,” “one implementation,” and/or “an implementation,” means that a feature, structure, or characteristic described in connection with the embodiment/implementation can be included in at least one embodiment/implementation. Thus, the appearances of such a phrase “in one embodiment,” “in an implementation,” etc. in various places throughout this specification are not necessarily all referring to the same embodiment/implementation. Furthermore, the features, structures, or characteristics may be combined in any suitable manner in one or more embodiments/implementations.

As used herein, the terms “employing” or “employed by” can refer to an element (e.g., a hardware device) that is currently being employed, that has already been employed and/or that is to be employed.

As used herein, the term “entity” can refer to a machine, device, smart device, component, hardware, software, and/or human. A “user entity,” “client entity” or “administrative entity” can refer to an entity that employs one or more outputs of a system described herein for personal, public, consumer, business, and/or commercial use. that stores and accesses data/metadata at a network access storage system.

As used herein, the term “group” can refer to one or more.

A “group of hardware” or “equipment” can refer to a subset of hardware devices of an operation system, which hardware devices can comprise, but are not limited to, storage nodes, switch nodes, server nodes and/or corresponding communication devices, and which operation system can comprise one or more computing systems.

As used herein, with respect to any aforementioned and below mentioned uses, the term “in response to” can refer to any one or more states including, but not limited to: at the same time as, at least partially in parallel with, at least partially subsequent to and/or fully subsequent to, where suitable.

As used herein, the term “power” can refer to electrical and/or other source of power available to the operation system.

As used herein, the term “resource” can refer to power, money, memory, CPU bandwidth, processing power, labor, hardware, and/or software.

As used herein, the term “set” can refer to one or more.

### Example Architectures

One or more embodiments are now described with reference to the drawings, where like referenced numerals are used to refer to like elements throughout. In the following description, for purposes of explanation, numerous specific details are set forth to provide a more thorough understanding of the one or more embodiments. It is evident, however, in various cases, that the one or more embodiments can be practiced without these specific details.

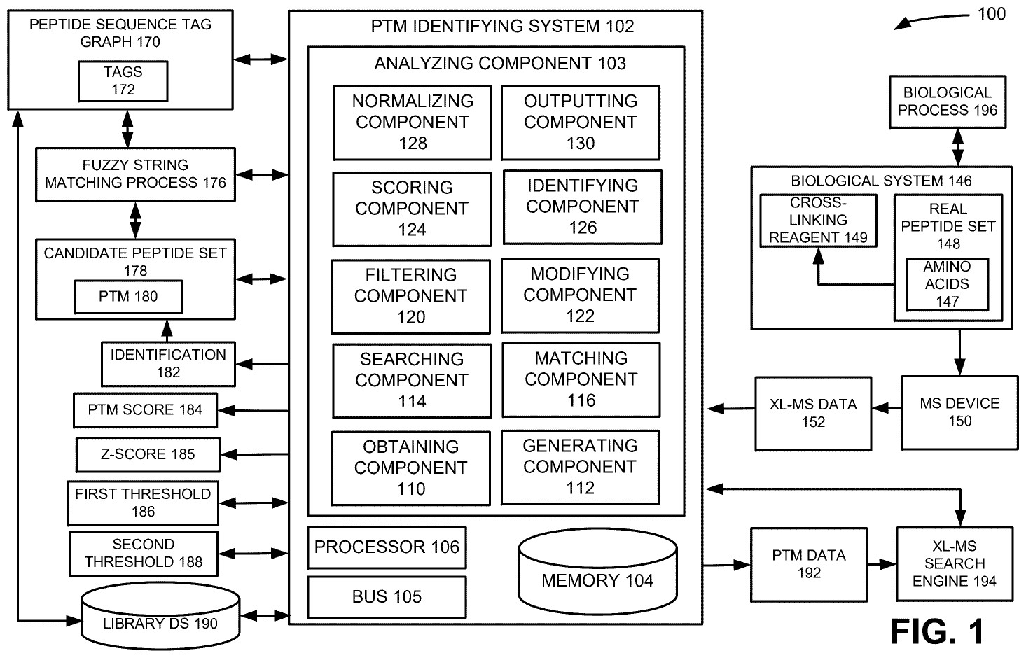

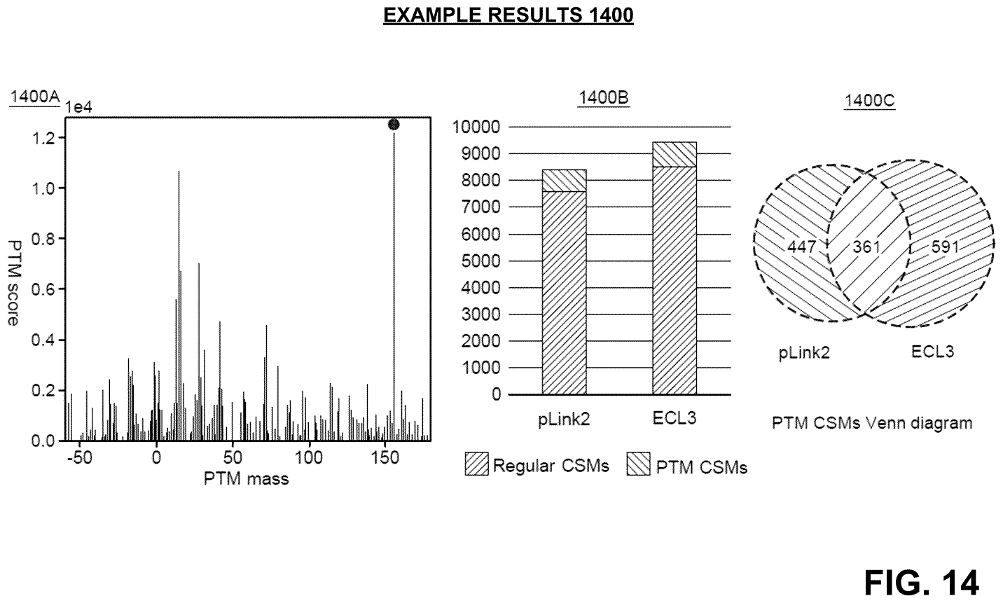

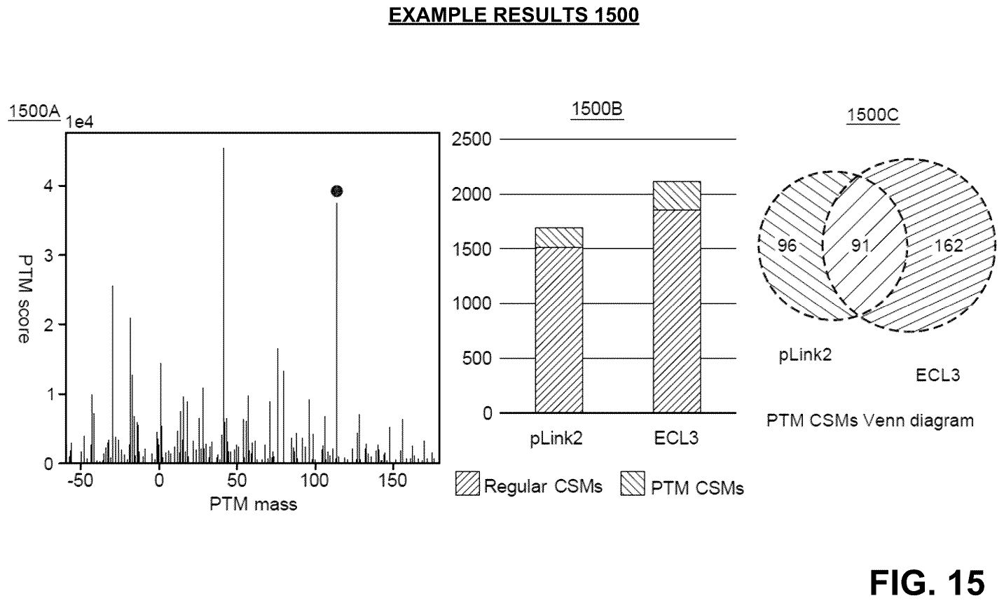

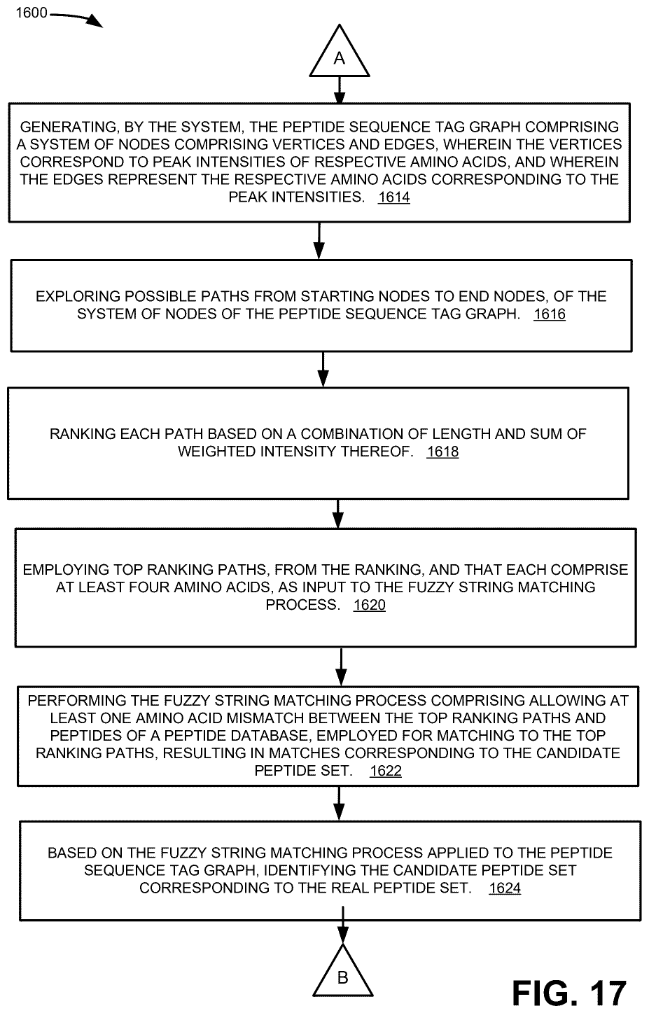

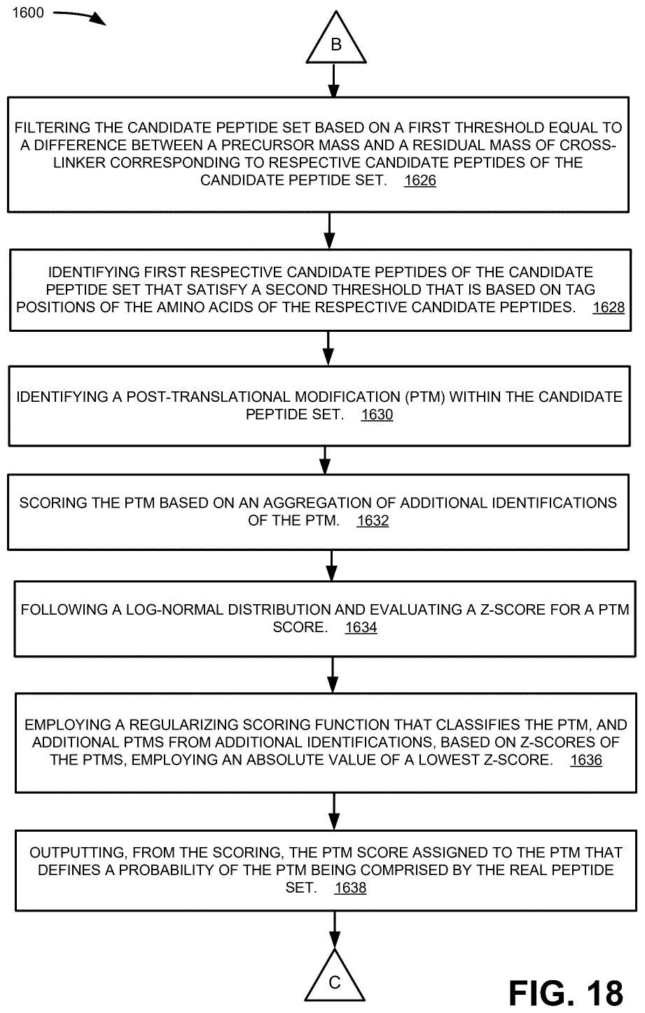

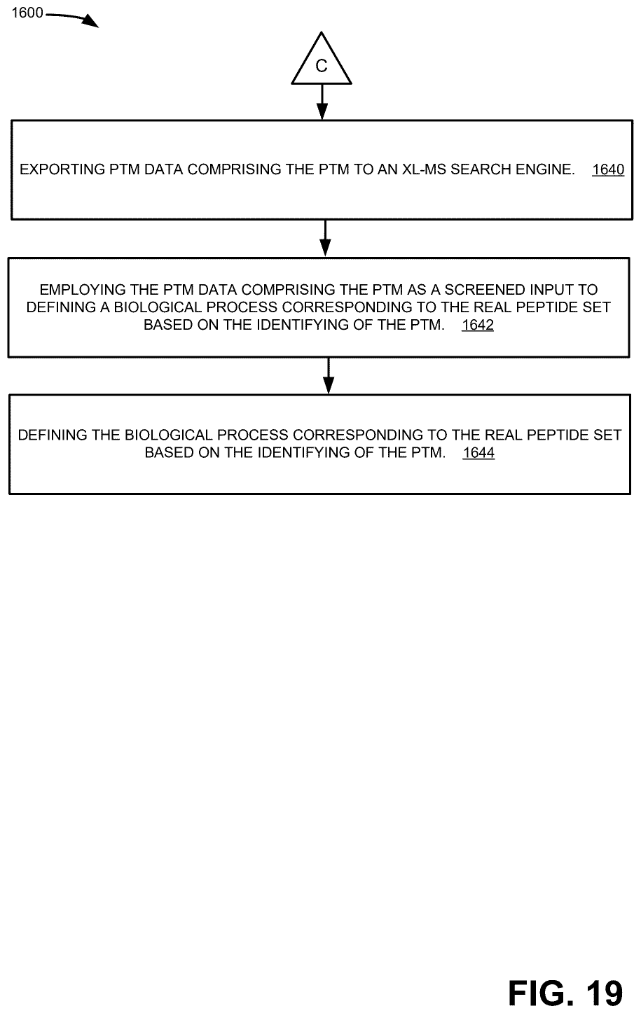

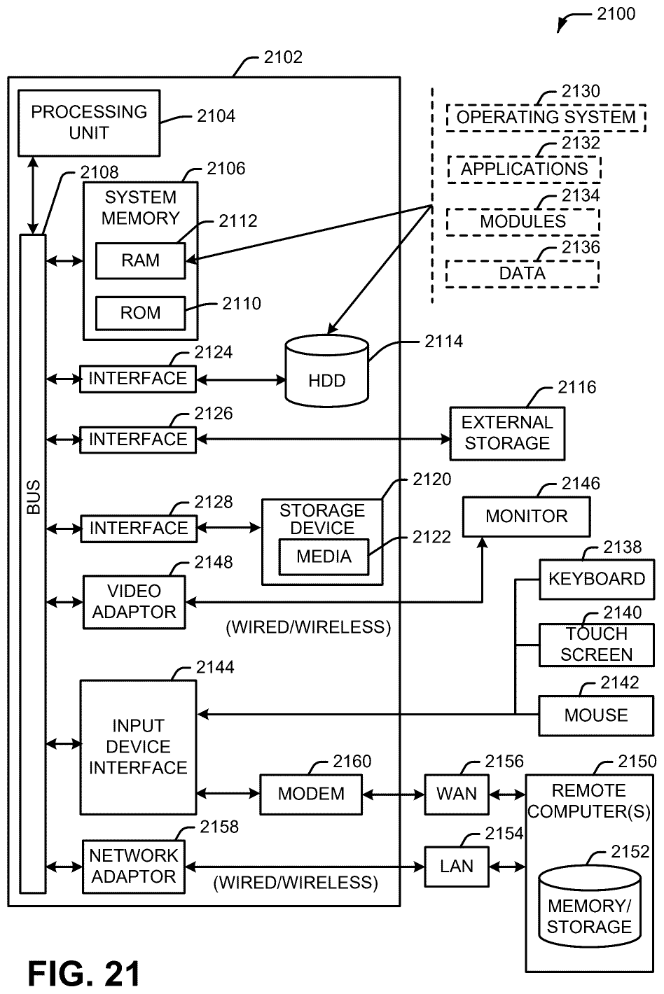

Further, the embodiments depicted in one or more figures described herein are for illustration only, and as such, the architecture of embodiments is not limited to the systems, devices and/or components depicted therein, nor to any order, connection and/or coupling of systems, devices and/or components depicted therein. For example, in one or more embodiments, the non-limiting system architectures described, and/or systems thereof, can further comprise one or more computer and/or computing-based elements described herein with reference to an computing environment, such as the computing environment 2100 illustrated at FIG. 21. In one or more described embodiments, computer and/or computing-based elements can be used in connection with implementing one or more of the systems, devices, components, and/or computer-implemented operations shown and/or described in connection with FIGS. 1-27 and/or with other figures described herein.

Turning now to FIG. 1, a non-limiting system 100 is illustrated that can comprise a PTM identifying system 102, a scientific analysis system, such as an MS device 150, and a library datastore (DS) 190.

In one or more embodiments, the MS device 150, such as a spectrometry device or spectrometer, can be separate from but communicatively couplable to the non-limiting system 100. In one or more other embodiments, the MS device 150 can comprise the PTM identifying system 102.

In one or more embodiments, one or more additional scientific analysis systems likewise can be communicatively couplable with the non-limiting system 100 and/or comprised by the non-limiting system 100, such as a chromatography device.

In one or more embodiments, the library datastore 190 can be separate from, but communicatively couplable to, the non-limiting system 100.

Generally, the PTM identifying system 102 can facilitate generation of a peptide sequence tag graph 170, and based thereon, identification and scoring of one or more PTMs 180 (e.g., with PTM scores 184). This data can be employed directly and/or can be employed as input to an SL-MS search engine 194.

In one or more embodiments, an XL-MS search engine 194 can be comprised by the non-limiting system 100. In one or more embodiments, an XL-MS search engine 194 can be separate from but communicatively couplable to the non-limiting system 100.

One or more communications between one or more components of the non-limiting system 100 can be provided by wired and/or wireless means including, but not limited to, employing a cellular network, a wide area network (WAN) (e.g., the Internet), and/or a local area network (LAN). Suitable wired or wireless technologies for supporting the communications can include, without being limited to, wireless fidelity (Wi-Fi), global system for mobile communications (GSM), universal mobile telecommunications system (UMTS), worldwide interoperability for microwave access (WiMAX), enhanced general packet radio service (enhanced GPRS), third generation partnership project (3GPP) long term evolution (LTE), third generation partnership project 2 (3GPP2) ultra-mobile broadband (UMB), high speed packet access (HSPA), Zigbee and other 802.XX wireless technologies and/or legacy telecommunication technologies, BLUETOOTH®, Session Initiation Protocol (SIP), ZIGBEE®, RF4CE protocol, WirelessHART protocol, 6LoWPAN (Ipv6 over Low power Wireless Area Networks), Z-Wave, an advanced and/or adaptive network technology (ANT), an ultra-wideband (UWB) standard protocol and/or other proprietary and/or non-proprietary communication protocols.

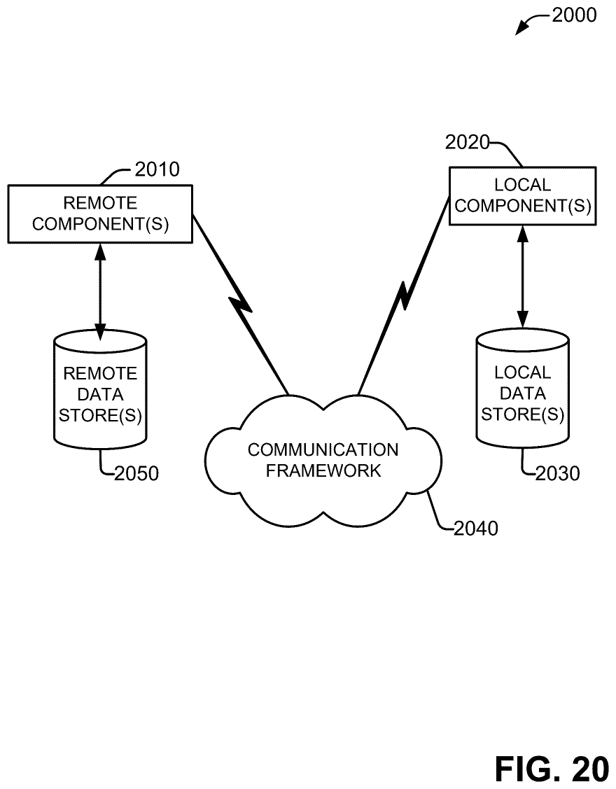

The PTM identifying system 102 can be associated with, such as accessible via, a cloud operating environment, such as the cloud operating environment 2000 of FIG. 20.

The PTM identifying system 102 can comprise a plurality of components. The components can comprise a memory 104, processor 106, bus 105, obtaining component 110, generating component 112, searching component 114, matching component 116, filtering component 120, modifying component 122, scoring component 124, identifying component 126, normalizing component 128 and/or outputting component 130. Using these components, the PTM identifying system 102 can perform the peptide sequence tag graph 170 generation, subsequent analysis processes using the peptide sequence tag graph 170, and PTM 180 identification and scoring thereafter.

Discussion next turns to the processor 106, memory 104 and bus 105 of the PTM identifying system 102. For example, in one or more example embodiments, the PTM identifying system 102 can comprise the processor 106 (e.g., computer processing unit, microprocessor, classical processor, quantum processor and/or like processor). In one or more example embodiments, a component associated with PTM identifying system 102, as described herein with or without reference to the one or more figures of the one or more example embodiments, can comprise one or more computer and/or machine readable, writable and/or executable components and/or instructions that can be executed by processor 106 to provide performance of one or more processes defined by such component and/or instruction. In one or more example embodiments, the processor 106 can comprise one or more of the obtaining component 110, generating component 112, searching component 114, matching component 116, filtering component 120, modifying component 122, scoring component 124, identifying component 126, normalizing component 128 and/or outputting component 130.

In one or more example embodiments, the PTM identifying system 102 can comprise the computer-readable memory 104 that can be operably connected to the processor 106. The memory 104 can store computer-executable instructions that, upon execution by the processor 106, can cause the processor 106 and/or one or more other components of the PTM identifying system 102 (e.g., obtaining component 110, generating component 112, searching component 114, matching component 116, filtering component 120, modifying component 122, scoring component 124, identifying component 126, normalizing component 128 and/or outputting component 130) to perform one or more actions. In one or more example embodiments, the memory 104 can store computer-executable components (e.g., obtaining component 110, generating component 112, searching component 114, matching component 116, filtering component 120, modifying component 122, scoring component 124, identifying component 126, normalizing component 128 and/or outputting component 130).

The PTM identifying system 102 and/or a component thereof as described herein, can be communicatively, electrically, operatively, optically and/or otherwise coupled to one another via a bus 105. Bus 105 can comprise one or more of a memory bus, memory controller, peripheral bus, external bus, local bus, quantum bus and/or another type of bus that can employ one or more bus architectures. One or more of these examples of bus 105 can be employed.

In one or more example embodiments, the PTM identifying system 102 can be coupled (e.g., communicatively, electrically, operatively, optically and/or like function) to one or more external systems (e.g., a non-illustrated electrical output production system, one or more output targets and/or an output target controller), sources and/or devices (e.g., classical and/or quantum computing devices, communication devices and/or like devices), such as via a network. In one or more example embodiments, one or more of the components of the PTM identifying system 102 and/or of the non-limiting system 100 can reside in the cloud, and/or can reside locally in a local computing environment (e.g., at a specified location).

In addition to the processor 106 and/or memory 104 described above, the PTM identifying system 102 can comprise one or more computer and/or machine readable, writable and/or executable components and/or instructions that, when executed by processor 106, can provide performance of one or more operations defined by such component and/or instruction.

Discussion next turns to the additional components of the PTM identifying system 102 (e.g., obtaining component 110, generating component 112, searching component 114, matching component 116, filtering component 120, modifying component 122, scoring component 124, identifying component 126, normalizing component 128 and/or outputting component 130).

Processes performed by the PTM identifying system 102 can generally be broken down into various sets of processes including, but not limited to a first set of processes for generation of a peptide sequence tag graph 170 based on XL-MS data 152, a second set of processes for analyzing the peptide sequence tag graph 170, and a third set of processes for identification and scoring of one or more PTMs 180 based on the analyzing of the peptide sequence tag graph 170.

First, it is noted that in one or more example embodiments, the obtaining component 110, generating component 112, searching component 114, matching component 116, filtering component 120, modifying component 122, scoring component 124, identifying component 126, normalizing component 128 and/or outputting component 130 can be implemented independently, without one or more other of the obtaining component 110, generating component 112, searching component 114, matching component 116, filtering component 120, modifying component 122, scoring component 124, identifying component 126, normalizing component 128 and/or outputting component 130. Additionally and/or alternatively, the obtaining component 110, generating component 112, searching component 114, matching component 116, filtering component 120, modifying component 122, scoring component 124, identifying component 126, normalizing component 128 and/or outputting component 130 can be comprised by a high-level analyzing component 103, one or more of the below-described functions of the obtaining component 110, generating component 112, searching component 114, matching component 116, filtering component 120, modifying component 122, scoring component 124, identifying component 126, normalizing component 128 and/or outputting component 130 can be performed by the high-level analyzing component 103, and/or obtaining component 110, generating component 112, searching component 114, matching component 116, filtering component 120, modifying component 122, scoring component 124, identifying component 126, normalizing component 128 and/or outputting component 130 can be omitted with the high-level analyzing component 103 performing one or more of the below-described functions of the one or more omitted obtaining component 110, generating component 112, searching component 114, matching component 116, filtering component 120, modifying component 122, scoring component 124, identifying component 126, normalizing component 128 and/or outputting component 130.

As noted above, a first set of one or more processes can comprise generation of a peptide sequence tag graph 170 based on XL-MS data 152.

Turning first to the obtaining component 110, this component can generally acquire (e.g., obtain, download, upload, request, transmit, etc.) XL-MS data 152 from a scientific measurement system, such as the MS device 150. In one or more cases, the XL-MS data 152 can correspond to a real peptide set 148, in a biological system 146, cross-linked by a non-cleavable cross-linking reagent or a cleavable cross-linking reagent 149. The obtaining component 110 can store this XL-MS data 152 either temporarily and/or more permanently at the library datastore 190, for example, in any suitable format. The XL-MS data 152 can comprise data and/or metadata in any suitable format.

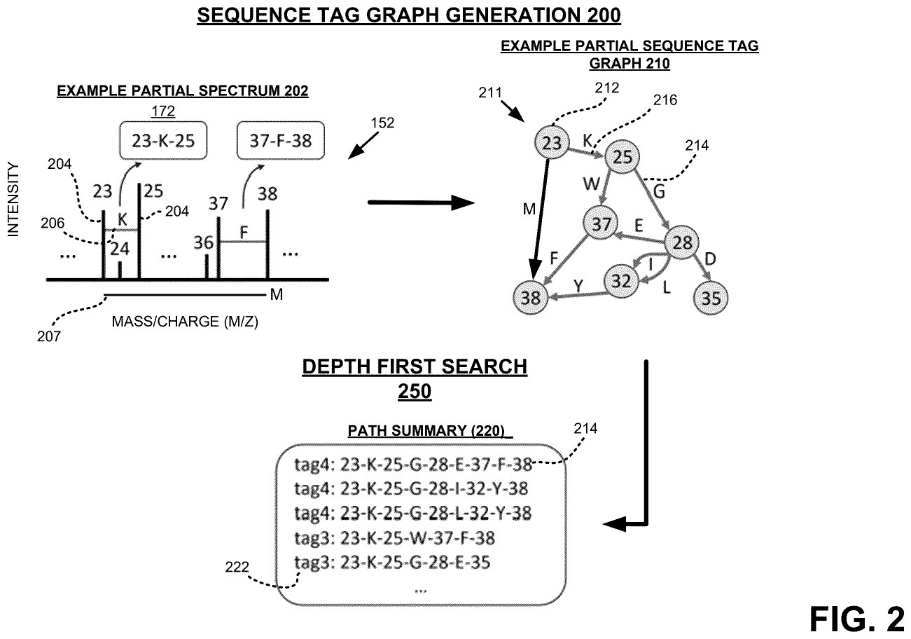

Turning to FIG. 2, using the XL-MS data 152, the generating component 112 can generally generate a sequence tag graph 210 using a sequence tag graph generation process 200.

First, in one or more embodiments, as part of the sequence tag graph generation process 200, the generating component 112 can de-charge the XL-MS data 152 into an alternate file format defining at least precursor mass, charge and mass/charge abundance pairs. For example, XL-MS data 152 can have higher charge states and more complex isotopic clusters in MS2 spectra than conventional MS data (e.g., non-cross-linking mass spectroscopy data). In one or more cases, the alternate file format can be a mascot generic file (MGF) format, or another suitable file format employed in proteomics, for example.

With respect to FIG. 2, and referring in particular to the example partial spectrum 202, to generate the tag graph 210, the generating component 112 can generally identify mass differences 206 between pairs of peaks 204, of the XL-MS data 152 along the mass/charge (m/z) axis (shown as the X-axis at the example partial spectrum 202), as corresponding to masses of known amino acids. More particularly, the generating component 112 can determine mass differences between all sets of peaks 204, and not only those adjacent to one another at an intensity vs. m/z graph of the XL-MS data 152. The generating component 112 can further determine whether all pairs of all peaks 204 of the XL-MS data 152 have been identified and whether all mass differences 206 corresponding to all of the pairs of peaks 204 have been identified. For example, at the example partial spectrum 202 of FIG. 2, a mass difference 206 between peaks 23 and 25 can represent an amino acid K, a mass difference 206 between peaks 27 and 28 can represent an amino acid F, and/or a mass difference 206 between peaks 23 and 38 can represent an amino acid M.

It is noted that mass differences between all pairs of peaks 204 do not all represent amino acids 147. Rather, the generating component 112 can identify for every two peaks 204 if the mass different 206 therebetween matches and/or corresponds to any of the known twenty amino acids' masses. In one or more cases a selected error tolerance can be employed for the values, such as a user-defined error tolerance and/or a default error tolerance of the mass difference 206 relative to the known masses of the known amino acids.

Subsequently, based on this identifying, the generating component 112 can generate tags 172 having respective lengths of one amino acid 147 and a peptide sequence tag graph 170 comprising the tags 172. For example, a tag 23-K-25 can correspond to the amino acid K, a tag 37-F-38 can correspond to the amino acid F, and/or a tag 23-M-38 can correspond to the amino acid M.

Put another way, if a match is found between a mass difference 206 and a known amino acid 147, such as based on a database of known amino acids at a datastore, such as the library datastore 190, the location of the corresponding peaks 204 and the corresponding amino acid information can be recorded as a tuple or tag 172 of amino acid length of one, by the generating component 112. For example, a tag 172 can have the form of p1-aa-p2, where p1 represents the location of the lighter mass peak, aa represents the amino acid letter, and p2 represents the location of the heavier mass peak. For instance, looking to the example partial spectrum 202 of FIG. 2, 23-K-25 indicates that the 23rd and 25th peaks in the spectra form a lysine mass. This tuple with a length of one amino acid is referred to as tag1.

Using these tags 172, the generating component 112 can aggregate and/or combine the tags 172 as the peptide sequence tag graph 170 based on the dataset of XL-MS data 152 and representing a real peptide set 148. The peptide sequence tag graph 170 can comprise a set of nodes 211 having vertices 212 and edges 216, where common vertices 212 from the tags 172 can have one or more edges 216 extending therefrom. That is, the vertices 212 can correspond to peak intensities of respective amino acids (e.g., comprised by the peptides of the real peptide set 148), and the edges 216 can represent the respective amino acids 148 corresponding to the peak intensities.

Put another way, once the extraction of all tag1 information is completed, the directed graph (e.g., the sequence tag graph 170) can be generated using these tags. In the sequence tag graph 170, each vertex 216 can represent the location (e.g., intensity location and/or mass/charge location) of a peak 204, and each edge 216 can represent information corresponding to a respective amino acid 147. For example, as illustrated at the example partial sequence tag graph 210 of FIG. 2, the peaks 23, 25, 28, 32, 35, 37 and 38 are illustrated with edges 216 extending therebetween. The edges 216 correspond to the mass differences 206 for which amino acids were matched.

Discussion next turns to the searching component 114 and to the depth first search 250.

After constructions of the sequence tag graph 170 by the generating component 112, the searching component 114 can proceed to identify paths 214, such as all possible paths 214, comprised by the sequence tag graph 170. The searching component 114 can analyze graph data defining the sequence tag graph 170 exploring possible paths (e.g., paths 214) from starting nodes to end nodes, of the system of nodes of the peptide sequence tag graph.

For example, as illustrated at FIG. 2, a path summary 220 can comprise a list of paths 214 comprised by the example partial sequence tag graph 210 of FIG. 2. As one path 214, the first tag4 follows a path from peak 23 to peak 38, including amino acids K, G, E and F.

Put another way, the searching component 114 can analyze graph data defining the sequence tag graph 170, such as by starting with vertices 216 having no incoming edges 216 and treating such vertices 216 as starting nodes of the nodes 211. Using a depth-first search (DFS) approach, the searching component 114 can explore possible paths 214, such as all possible paths 214, from these starting nodes to the end nodes in the sequence tag graph 170.

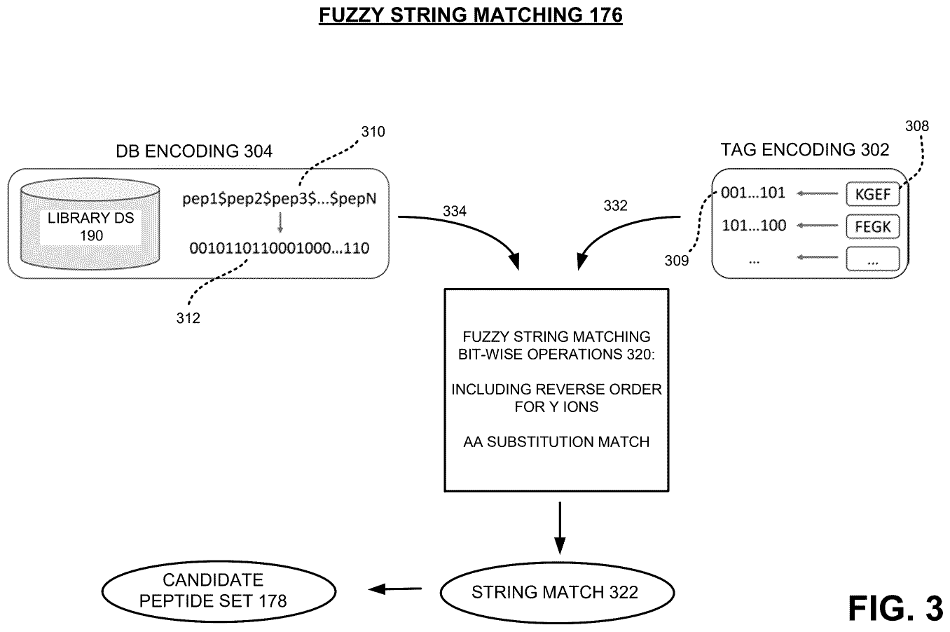

Turning next to FIG. 3, a fuzzy string matching process 176 can be performed by the PTM identifying system 102. The fuzzy string matching process 176 can comprise sub-processes that can be performed prior to a main fuzzy string matching. These sub-processes can comprise database encoding, tag encoding, path ranking and/or path filtering.

For example, as illustrated at FIG. 3, database encoding 304 can comprise concatenating known peptides using the “$” sign as separating symbol. The concatenated peptides can be encoded into binary form or another suitable form for comparison against the tags 172. Tag encoding 302 can comprise encoding the tags 172 into binary form or another suitable form for comparison against the peptides of the peptide database 190.

The paths 214 generally can be pruned by the matching component 116, merging similar paths to enhance robustness of results from the fuzzy string matching process 176.

In one or more pruning cases, within a path 214, if a vertex 212 has a significantly lower peak intensity (e.g., less than 10%) compared to its neighboring vertices 212 (neighboring within the graph 170), this vertex 212 can be removed from the path 214. This action breaks the original path into two sub-paths.

In one or more pruning cases, pairs of paths 214, such as all pairs of paths 214, can be compared, such as against a threshold, such as a 70% overlap threshold. In one example, if there is 70% or greater overlap, or the threshold is satisfied, a new path 214 can be generated using the overlap, along with deletion and/or removal of data corresponding to the original two overlapping paths 214. This merging process can aid in eliminating redundant information and streamline the results output from the fuzzy string matching process 176.

Ranking can be performed by the matching component 116, such as for each path 214,

In one or more ranking cases, paths can be ranked based on length of path 214, sum of weighted intensity of path 214, or on a combination of length and sum of weighted intensity thereof. A weighted intensity can be calculated by dividing a total intensity of a tag 172 by the length of the tag 72. The top N (default 10) paths are retained for further analysis.

As noted, another sub-process can comprise employing top ranking paths 214, from the ranking, and that each comprise at least four amino acids 147 (e.g., at least one tag 4), as input to a fuzzy string matching model, algorithm, sub-process, etc. This can ensure significance and/or relevance.

Additionally, and/or alternatively, a top set of N tags can be identified for input into the fuzzy string matching process 176, where N is a default or user-selected quantity.

It is noted that any one or more of the pruning sub-processes and/or the ranking sub-processes can be performed at least partially in parallel with one another. One or more pruning sub-processes can be performed prior to one or more ranking sub-processes, or vice versa.

Discussion turns now to the main fuzzy string matching. Given the top paths 214, such as the top N paths 214, derived from the graph 170, the matching component 116 can generally determine one or more corresponding known peptide sequences that match the encoded tags (e.g., such as of the path summary 220).

In one or more cases, the matching component 116 can perform an in silico protein digestion, such as without considering decoy proteins, and can subsequently combine all peptide sequences into a single string (e.g., peptide sequences resulting from tag encoding 302 and/or database encoding 304.

A bitap (shift-or) algorithm, model and/or process can be employed by the matching component 116 for the string matching to search for tag patterns between the tag encoded data 332 and the database encoded data 334. In one or more cases, this matching can comprise a bit-wise operation 320 of including reverse order for y series ions in the database. In one or more additional and/or alternative cases, this matching can comprise a bit-wise operation 320 of allowing for one amino acid substitution mismatch between a portion of the database encoded data 334 and the tag encoded data 332.

This implementation can be efficient and can exhibit a predictable running time complexity of O (mn), where m represents the length of the concatenated peptide and n represents the length of a respective tag pattern.

As a result of the above one or more processes and/or sub-processes, the fuzzy string matching 176 can result in one or more string matches 322 that together can define a candidate peptide set 178.

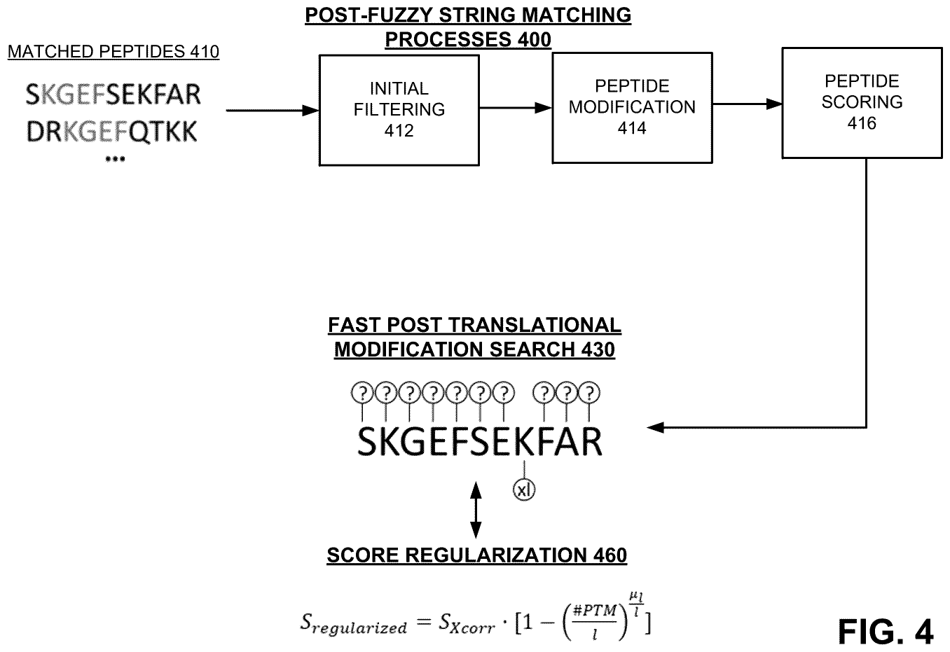

Discussion next turns to FIG. 4 and to one or more post-fuzzy string matching processes that can comprise one or more of initial filtering 412, peptide modification 414, and/or peptide scoring 416.

Initial filtering 412 can generally comprise filtering, by the filtering component 120, of the candidate peptide set based on a first threshold 186 equal to a difference between a precursor mass and a residual mass of a cross-linker (e.g., cross-linking reagent 149) corresponding to respective candidate peptides of the candidate peptide set 178. Put another way, in one or more cases, the observed peptide mass of an acceptable candidate peptide should not exceed the precursor mass minus the residual mass of the cross-linker. If exceeding, the candidate peptide is filtered out of the candidate peptide set 178.

Additionally, or alternatively, initial filtering 412 can comprise identifying, by the filtering component 120, first respective candidate peptides of the candidate peptide set 178 that satisfy a second threshold 188 that is based on tag positions of the amino acids 147 of the first respective candidate peptides. Put another way, if theoretical peaks for the tag position in the peptide sequence deviate significantly from the corresponding experimental peaks (i.e., outside the mass tolerance range of ±250 Dalton), the respective candidate peptide can be considered invalid and is filtered out of the candidate peptide set 178.

Peptide modification 414 can generally comprise modifying, by the modifying component 122, mass of a peptide, more in line with a real peptide to allow for an alignment of a candidate peptide to the real peptide. For example, to achieve perfect alignment between theoretical peaks and tag peaks in the MS2 spectrum, mass at the N-terminus of the observed peptide (corresponding to the graph data of the peptide sequence tag graph 170) can be adjusted, such as by the modifying component 122, for b ions tags (or C-terminus for y ions tags), resulting in a modified peptide mass.

Additionally or alternatively, additional mass can be introduced, such as by the modifying component 122, at a link site of an observed peptide, specifically equal to a difference between the respective precursor mass and the modified peptide mass obtained. This adjustment can lead to the formation of a potential cross-linked peptide.

Peptide scoring 416 can comprise assigning scores, by the scoring component 124, to observed peptides, such as modified cross-linked peptides, such as all observed peptides and/or all modified cross-linked peptides. Scoring by the scoring component 124 can employ an Xcorr scoring function as understood by one having ordinary skill in the art. Top results, such as M top results, can be retained, such as where M is a default or user-defined number. Top results can be identified, such as by the scoring component 124, based on any suitable quantitative threshold, such as a default threshold or a user-defined threshold.

Any one or more of the initial filtering 412, peptide modification 414 and/or scoring processes 416 discussed above can be employed, and any one or more of these processes can be performed at least partially in parallel with one another. Additionally or alternatively, these processes can be performed in any order.

Next, discussion turns to fast post translational modification (PTM) search 430 and score regularization 460, while still referring to FIG. 4. As will be described below, score regularization can be performed by the identifying component 126 in conjunction with the fast PTM search 430, also performed by the identifying component 126.

Generally, the identifying component 126 can identify a post-translational modification (PTM) 180 within the candidate peptide set 178 (e.g., as comprised by fast PTM search 430).

A suitable searchable database can be employed, such as a Unimod database, comprising known PTMs. For example, a Unimod database can comprise approximately 1,5000 PTMs. Based on a comparison performed by the identifying component 126, the identifying component 126 can perform one or more PTM identifications 182. The comparison can comprise comparing data representing the candidate peptide set 178 with the Unimod database data, one or more PTMs present in the candidate peptide set 178 can be determined.

More specifically, a numerical evaluation can be performed, by the identifying component 126, of a total search space for a peptide sequence of length 1, using the selected database and the candidate peptide set 178. The numerical evaluation can comprise an assumption that on average, there could be k distinct PTMs 180 for each amino acid 147. When considering a single PTM case on a peptide, there are

$$( \frac{l}{1} ) * {k}^{1} = k * 1$$

different combinations. For two PTM cases, the number of combinations can be

$$( \frac{l}{2} ) * {k}^{2} ,$$

and so on, up to I FIM cases. The total number of PTMs that are to be considered by the identifying component 126 generally is equal to the term (1+k)^{l}−1. This term can grow exponentially with respect to l and polynomially with respect to k.

It will be appreciated that the search space for the identifying component 126 can be prohibitively large, making it challenging to explore thoroughly, such as in a timely and/or efficient manner. To address this issue, the PTM identifying system 102 can employ a fast PTM search method 430 that can generally limit the maximum number of combinations to

$$\frac{{k ( 1 + l )}^{l}}{2} ,$$

where this search space is a polynomial function of l and a linear function of k.

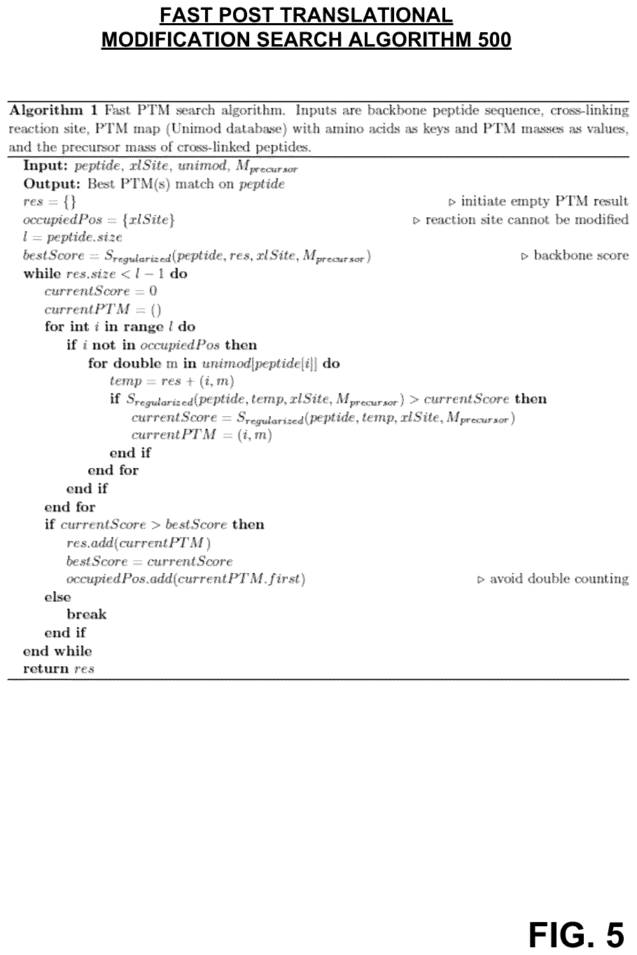

Turning briefly to FIG. 5, an example PTM search algorithm 500 is illustrated. Inputs can comprise the backbone peptide sequence, cross-linking reaction site, PTM map from the selected database with amino acids as keys and PTM masses as values, and the precursor mass of cross-linked peptides of the candidate peptide set 178.

The general concept can comprise, for each peptide candidate of the candidate peptide set 178, assuming that the peptide candidate represents one side of the cross-linked peptides and identifying the PTMs on this sequence. If multiple PTMs are present, matching the spectrum to the bald backbone sequence can yield a low score since only a limited number of peaks 204 can be matched. However, as true PTM information is gradually incorporated into the sequence one by one, the match result can gradually improve. This process can be continued, incrementally adding PTMs until the match results no longer show any significant improvement. By iteratively adding PTMs, the PTM space can be systematically explored, and the user can determine the optimal number and type of PTMs in a maximum number of

$$\frac{{k ( 1 + l )}^{l}}{2}$$

combinations.

It is noted that the fast PTM search algorithm 500 can employ a regularized scoring function for identifying PTMs and/or nonprobable instances (not PTMs), provided below as Equation 1.

$${S}_{regularized} = {S}_{Xcorr} * [ 1 - {( \frac{\# PTM}{l} )}^{\frac{{μ}_{l}}{l}} ] . \qquad Equation 1$$

At Equation 1, S_{Xcorr} represents the original Xcorr scoring function, #PTM denotes the number of PTMs being tested, l denotes the length of peptide, and μₗ represents the average length of peptide candidates for the given experimental spectrum. This scoring function penalizes the occurrence of PTMs on the peptide sequence. In connection therewith, it can be observed that a higher number of PTMs on the peptide corresponds to a lower frequency of occurrence.

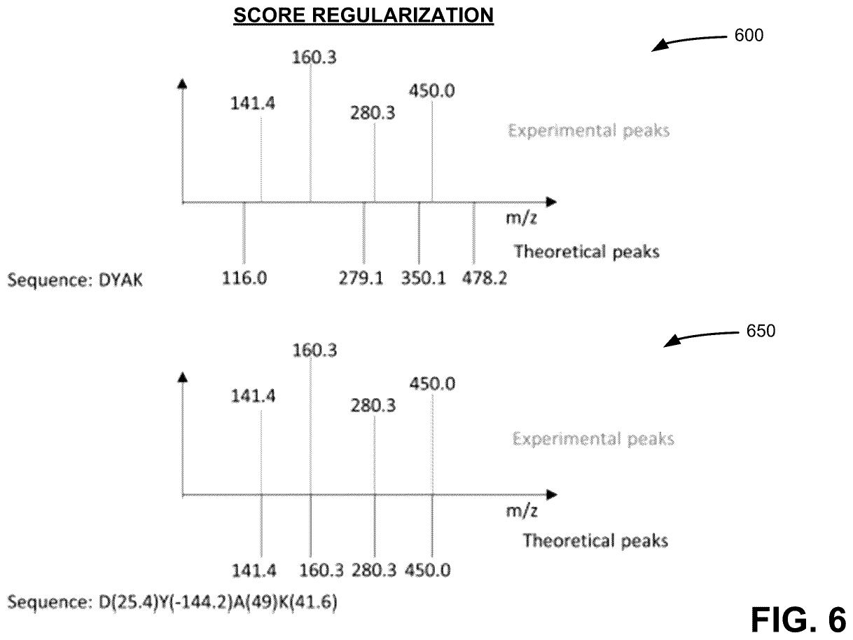

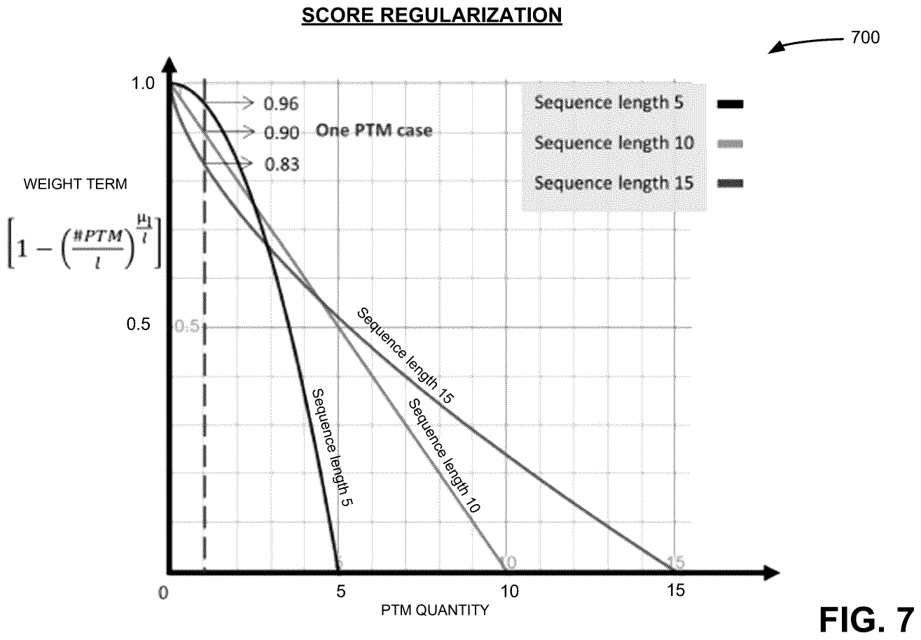

Still referring to PTM identification 182, and turning briefly to FIGS. 6 and 7, illustrated are illustrations 600 and 650 and graph 700. Illustrations 600 and 650 correspond to rationale behind the scoring function of Equation 1 and/or use at the fast PTM search algorithm 500. Graph 700 demonstrates the property of the weight term in the scoring function of Equation 1.

In particular, when considering probability of PTM occurrence within a peptide sequence, it is not always true that a better match of peaks leads to a better result. When there is no penalty for PTM occurrence within the peptide sequence, it has been observed that PTMs can be matched even when they do not exist in reality. This can be because adding (fake) PTMs to the sequence can result in a higher chance for the mismatched fragmented ions to match the noisy peaks in the spectrum.

To illustrate an extreme case, referring to FIG. 6, a scenario is considered where an incorrect backbone sequence is employed, where the incorrect backbone sequence does not have any theoretical ions matching the peaks in the spectrum. However, by purposely adding PTMs to each amino acid in this backbone sequence, new theoretical ions can be generated that match the peaks in the spectrum. This is possible if there are no limitations on the choice of PTMs, and their mass can be any real number. Therefore, a penalty is added in the scoring function to restrict the occurrence of the PTMs.

Put another way, FIG. 6 illustrates an extreme case of fake PTMs on each amino acid to perfectly match the peaks in the spectrum. The sequence DYAK cannot match any experimental peaks in the MS2 spectra. However, if fake PTMs are added with masses of 25.4 Da,−144.2 Da, 49 Da, and 41.6 Da on amino acids D, Y, A and K, respectively, the theoretical peaks can be caused to match the experimental ones perfectly.

Concretely, the scoring function is designed to satisfy several requirements. First, as the number of PTMs in the backbone sequence increases, the power should also gradually increase. Second, if there are no PTMs on the backbone sequence, there should not be any penalty for the peak matching. Third, if all amino acids are assigned PTMs, the score should be 0 regardless of how good the matching result is. Fourth, for peptides with different lengths, the penalty for a single PTM occurrence should vary.

Fifth, longer peptides should incur a higher penalty. The reason for this is a scenario with two peptide candidates: A, with a sequence length of 100, and B, with a sequence length of 10. Both sequences A and B have one identified PTM that increases their scores compared to their respective unmodified backbones' scores. Since A has 100 possible positions to add a PTM and improve the backbone score, while B only has 10 possible positions, A should be penalized more due to its higher random chance of achieving a better score.

Equation 1 is based on these five rationales, as provided above. Equation 1 is again provided below, along with its proof, for reference, as Equation 1′.

$${S}_{regularized} = {S}_{X corr} \cdot [ 1 - {( \frac{\# PTM}{l} )}^{\frac{{μ}_{l}}{l}} ] = \overline{e} \cdot \overline{t} \cdot [ 1 - {( \frac{\# PTM}{l} )}^{\frac{{μ}_{l}}{l}} ] . \qquad Equation 1 ’$$

At Equation 1′, S_{Xcorr} represents the original Xcorr scoring function, \vec{e} is the digitized experimental peaks, \vec{t} is the digitized theoretical peaks, #PTM denotes the number of PTMs, l denotes the length of peptide, and μₗ represents the average length of peptide candidates for the given spectrum.

To demonstrate the properties of the scoring function of Equation 1, especially for the weight term,

$$[ 1 - {( \frac{\# PTM}{l} )}^{\frac{{μ}_{l}}{l}} ] ,$$

reference is made to graph 700 of FIG. 7. Relative to graph 700, three peptide candidates are theorized for a specific spectrum, with lengths of 5, 10 and 15. Graph 700 illustrates the curves of the weight changes corresponding to these three lengths. As observed, the weight term satisfies the five aforementioned requirements.

That is, curves are generated at graph 700 that depict the relationship between the number of PTMs and the weight term in the regularized scoring function. First, all curves are decreasing functions, indicating that as the number of PTMs increases, the weight decreases. Secondly, each curve starts from the point (0,1), indicating that when there are no PTMs (PTM number equals 0), there is no penalty to the score. Third, every curve ends at the point (length, 0), indicating that when all amino acids are assigned PTMs (PTM number equals length), there is a maximum penalty resulting in a weight of 0. Lastly, at the position where the PTM number equals 1, the weight required decreases as the peptide length increases.

Finally, after matching each peptide with the best PTMs using the fast PTM search 430, the highest-scored peptide along with its corresponding PTMs 180 can be determined as one of the cross-linked peptides for the current spectrum (e.g., corresponding to the real peptide set 148), by the identifying component 126. However, if there are two peptides among the top candidates whose masses exactly add up to the precursor mass. In such cases, these two peptides are treated together as the best matches by the identifying component 126.

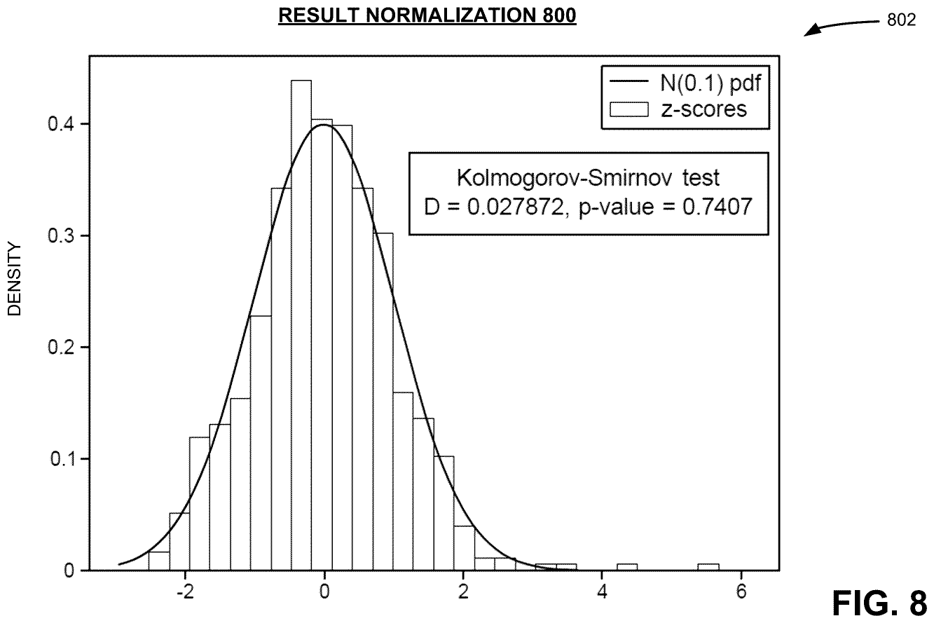

Discussion next turns to FIG. 8 and to result normalization 800 performed by the normalizing component 128. Generally the normalizing component 128 can score the PTM 180 based on an aggregation of additional identifications of the PTM 180, resulting in a PTM score 184 (e.g., as comprised by score regularization 460). That is, in one or more cases, this scoring can be performed, by the normalizing component 128, following a log-normal distribution and evaluating a z-score 185 for a PTM score 184. More particularly, in one or more cases, this scoring, by the normalizing component 128, can comprise employing a normalizing scoring function (Equation 2 below) that classifies the PTM 180, and additional PTMs from the additional identifications, based on the z-scores 185 of the PTMs 180, employing an absolute value of a lowest z-score 185 of the z-scores 185. Put another way, using the corresponding z-scores 185, the PTM score 184 assigned to the PTM 180 can define a probability of the PTM being comprised by the real peptide set 148.

Put another way, each identified PTM 180 can be assigned a PTM score 184 at Equation 2, which PTM score 184 aggregates peptide scores across all datasets where this PTM 180 has been identified.

$${Score}_{PTMi} = \sum {S}_{regularized} ( PTMi ) . \qquad Equation 2$$

At Equation 2, S_{regularized}(PTMi) represents the regularized score for the spectrum that identifies specific PTMi. These PTM scores 184 can follow a log-normal distribution. By taking the logarithmic form and calculating the z-score for each PTM's score, the normalizing component 128 can determine the relative significance of the PTM scores 184.

For example, histogram 802 at FIG. 8 illustrates observation on the distribution of z-scores 185, as compared with standard normal distribution. That is, the logarithmic form of PTM scores 184 can be converted into z-scores 185 by subtracting the mean and dividing by the standard deviation. The histogram of these z-scores can be plotted and compared, by the normalizing component 128, with the standard normal distribution using the K-S test. The p-value indicates that there are no significant differences.

PTM results can be classified into four categories based on the following criteria. The absolute value of the lowest z-score is set as the threshold (t). PTMs with z-scores between [1.2t, 1.5t) are considered moderate results, those between [1.5t, 2t) are considered good results, and those between [2t, +∞) are considered significant results. The remaining z-scores 185 are labeled as unsure or low probability.

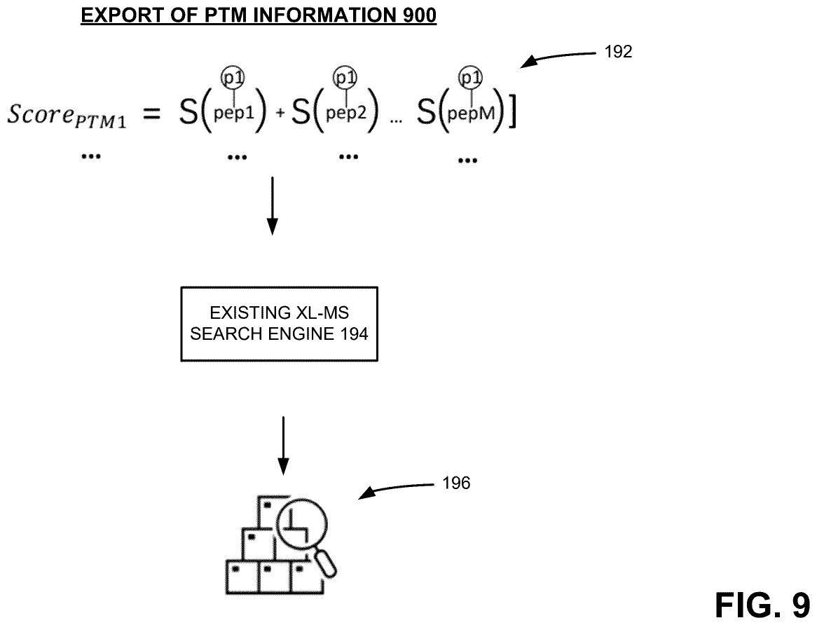

Now referring to FIG. 9, discussion next turns to export of PTM information 900. Generally, the outputting component 130 can output/export, from the scoring, PTM data 192 comprising the PTM 180 to an XL-MS search engine 194. This outputting and/or exporting can comprise any one or more of sending, transmitting, receiving, packaging, communicating, obtaining, uploading, downloading and/or the like using any suitable communication between the PTM identifying system 102 and the XL-MS search engine 194.

The purpose of the outputting can be for the PTM data 192 to serve as a variable modification to search for cross-linked peptides. When determining which PTMs should be included as variable modifications in an existing XL-MS search engine, it is recommended to consider them in order of high significance to lower significance, including as many as possible while ensuring a suitable running time for the specific search engine.

To achieve one or more of these operations, the outputting component 130 can direct the XL-MS search engine 194 to use the PTM data 192, or the XL-MS search engine 194, as part of the PTM identifying system 102 or separate from the PTM identifying system 102, and/or as part of the non-limiting system 100 or separate from the non-limiting system 100, can employ the PTM data 192.

In any of the above situations, the PTM data 192 can be employed as a screened input to defining a biological process 196 corresponding to the real peptide set 148 based on the identifying of the PTM 180. That is, the outputting component 130 and/or the XL-MS search engine 194 can define the biological process 196 corresponding to the real peptide set 148 based on the identifying of the PTM 180.

### Example Experimental Results

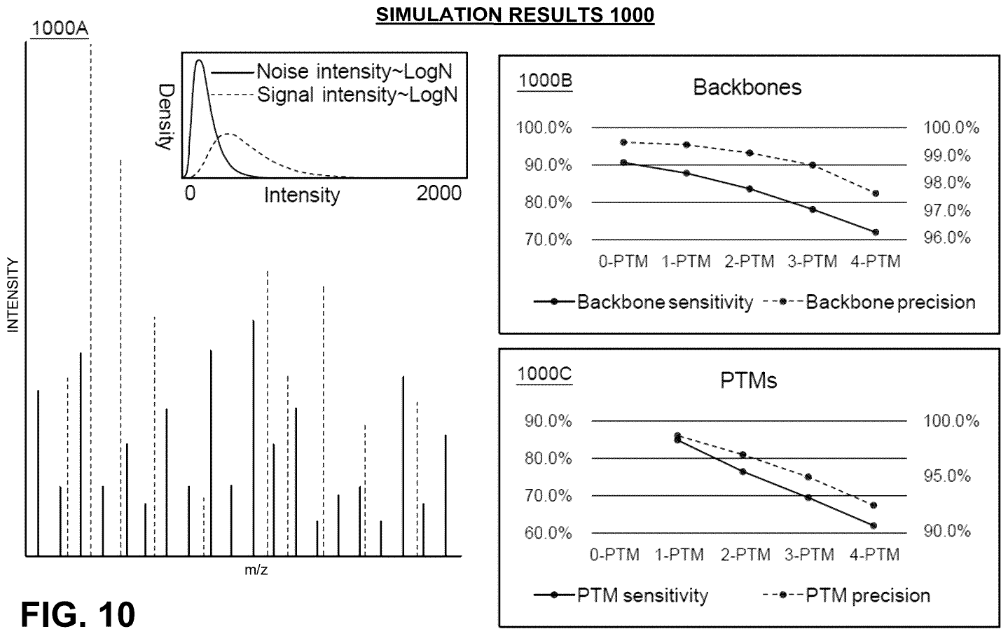

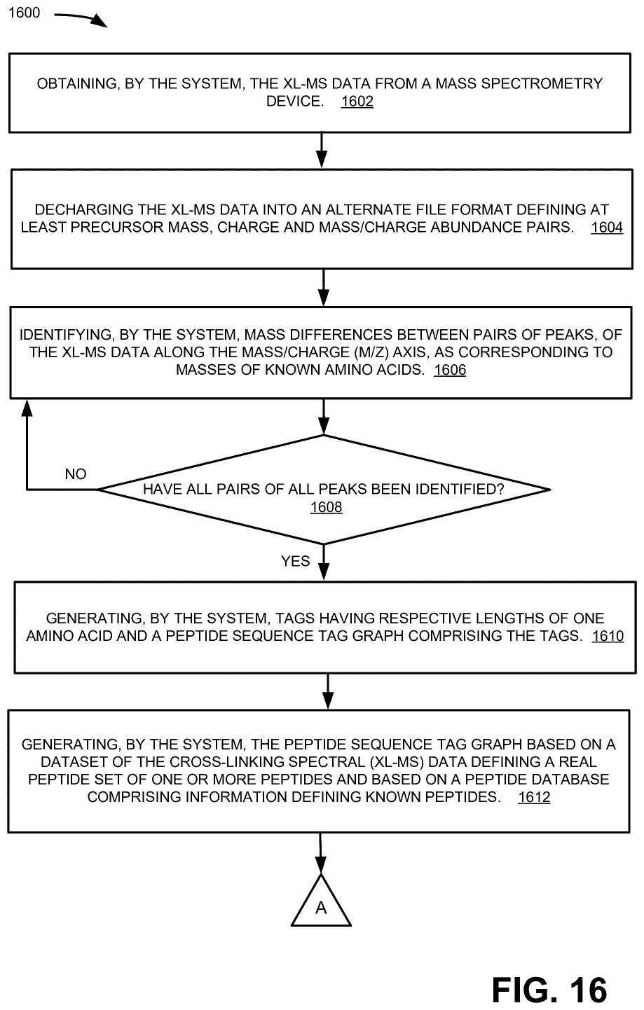

Turning next to FIGS. 10 to 15, illustrated are various graphs and/or datasets demonstrating various experimental and/or simulated results based on one or more aspects of the process flow 1600 (FIGS. 16 to 19), as can be performed by the non-limiting system 100.

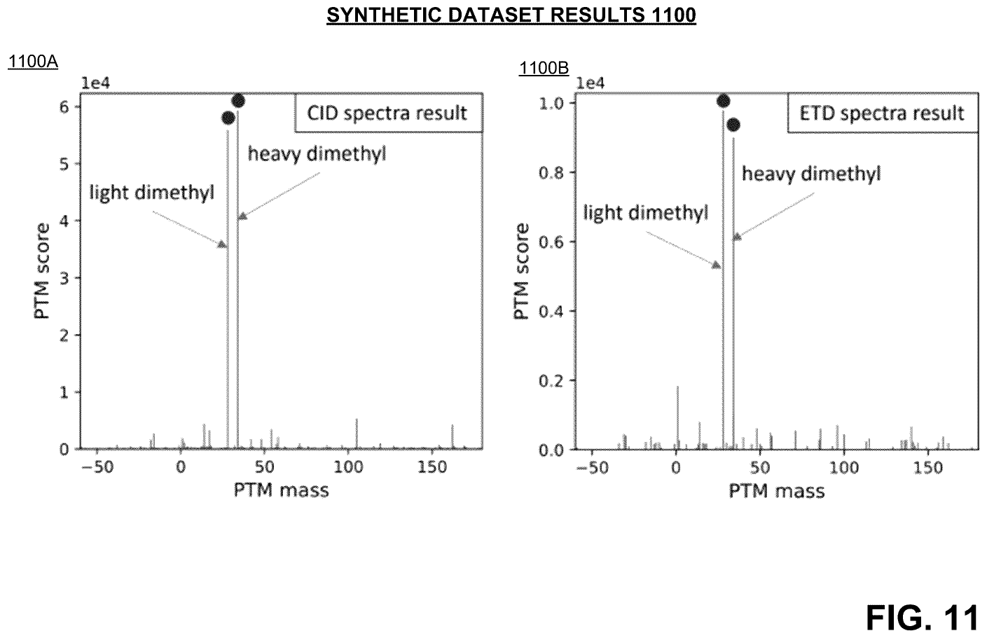

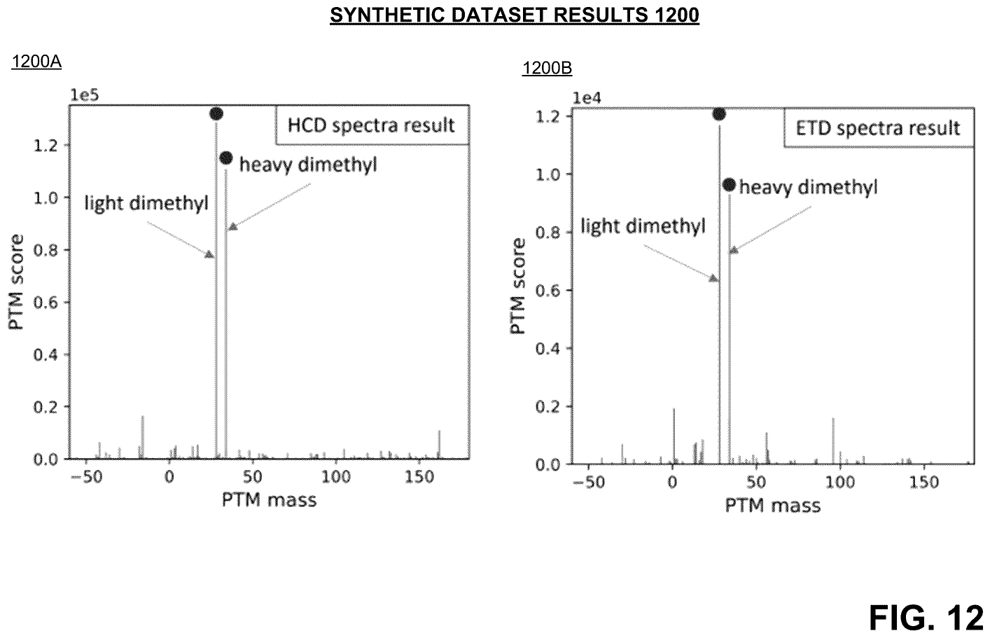

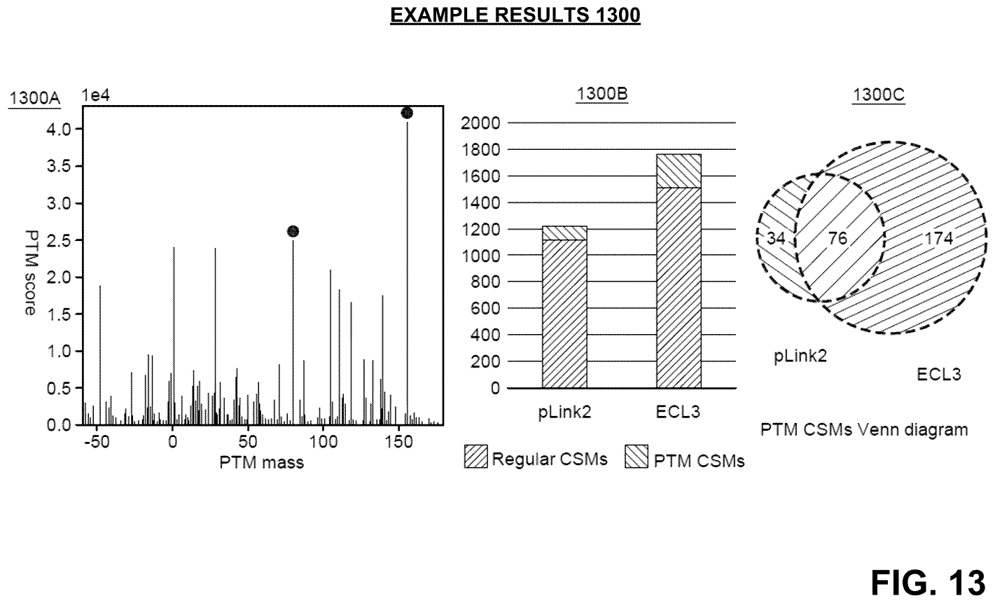

The FIGS. 10 to 15 are split between results based on simulated datasets (FIG. 10), synthetic isotope-labeled datasets (FIGS. 11 and 12), real experimental datasets (FIGS. 13 to 15), and linear peptide datasets (no associated figure), the results being based on different experiments employed to evaluate SeaPIC (e.g., the one or more embodiments described herein). Throughout the experiments, SeaPIC was tested for different types of spectra including CID, HCD, and ETD MS2 spectra. The experiments utilized the cleavable crosslinker disuccinimidyl sulfoxide (DSSO) and cyanurbiotindipropionylsuccinimide (CBDPS), and the non-cleavable cross-linker bissulfosuccinimidyl suberate (BS3), and bissulfosuccinimidyl glutarate (BS2G). SeaPIC was employed in conjunction with two different existing XL-MS search engines 194 to run out the final PTM-containing CSMs.

It is noted that all references both above and below to SeaPIC refer to one or more embodiments of the PTM identifying system 102.

### Simulated Datasets

Turning first to FIG. 10, illustrated are simulation results 1000, based on simulated datasets, and graphed at graphs 1000A, 1000B and 1000C.

For further information regarding the simulation experiment conducted that resulted in the simulation results illustrated at FIG. 10, initially conducted was a simulation experiment to evaluate the performance of SeaPIC. Approximately 100,000 spectra were simulated, each with varying numbers and categories of PTMs. SeaPIC was employed to interpret each simulated spectrum, assessing its ability to identify both the backbone sequences and the associated PTMs.

All E. coli proteins were first digested into peptides. For each simulation, two peptides were randomly selected from the E. coli database and assumed they were cross-linked peptides. Several PTMs were then introduced from the Unimod database onto these cross-linked peptides. The cleavable DSSO cross-linker was used to connect the two peptides. Next, MS2 spectra were generated based on these simulated PTM-containing cross-linked peptides. The MS2 spectra consist of both fragment (signal) peaks and noise peaks. Assumptions were made that both types of peaks followed a log-normal distribution, with signal peaks having higher intensity compared to noise peaks. Additionally, assumptions were made that noise peaks were uniformly distributed between 0 Dalton and the precursor mass. For each spectrum, the presence of 100 noise peaks was simulated. Graph 1000A shows an example of the simulated spectrum.

Graph 1000A provides a demonstration of the simulated spectrum. Two peptides were randomly selected from an E. coli database and several PTMs from a Unimod database. Given these artifact peptides, the fragment peaks (b/y ions) were generated with intensity following a log-normal distribution. Additionally, noise peaks were uniformly generated between 0 and the precursor mass, with intensity also following a log-normal distribution but at a lower level. Overall, 100,000 spectra were simulated with 0, 1, 2, 3, and 4 PTMs.

Generated were a total of 100,000 MS2 spectra, equally divided into five sets containing 0, 1, 2, 3, and 4 PTMs, respectively. SeaPIC was employed for the analysis of each dataset. While SeaPIC only provides PTM information as output without detailed scan information, the intermediate outcome was extracted from SeaPIC for each spectrum. Subsequently, this intermediate result was compared with the ground truth to determine the sensitivity and precision of the tool. Graphs 1000B and 1000C show the statistics for the backbone sequences and PTMs results.

In general, SeaPIC demonstrated a high sensitivity in accurately identifying the majority of backbone sequences and PTMs. SeaPIC can achieve a precision rate of over 90% across various scenarios involving different numbers of PTMs.

Graph 1000B illustrates the results for backbone sequences. Graph 1000B shows the performance of SeaPIC in detecting the backbone sequences. When there are no PTMs in the sequence, over 90% of the backbone sequences can be identified with nearly 100% precision. In the case of four PTMs in the peptide sequences, 72% of the backbones are identified with 97% precision.

That is, for the spectra set with no PTM, 90.6% of the cross-linked peptides are identified with 99.5% precision. As the number of PTMs increases, the performance of SeaPIC deteriorates, which is reasonable because the PTMs can affect the tag identification. Under the four PTMs situation, SeaPIC still identified 72.1% of the backbone sequences with 97.7% precision.

Graph 1000C depicts the results of PTM identification. Graph 1000C shows the performance of SeaPIC in detecting the PTMs in the sequences. For sequences with one PTM, 85% of the spectra can be identified with nearly 100% precision. When there are four PTMs in the peptide sequences, 62% of the PTMs are identified with 92% precision.

That is, in the spectra set with one PTM, 84.9% of the simulated spectra are identified with 98.6% precision. The detection ability also decreases with an increasing number of PTMs. When there are four PTMs in the spectrum, 62.1% of the results are identified with 92.5% precision. It can be observed that even in the four PTMs situation, SeaPIC still identified a big portion of PTMs.

In addition, employed were a pair of existing XL-MS search engines, pLink2 and ECL3, to analyze the aforementioned five datasets with default settings and observe their results. As shown in supplemental Table 1, except for the 0-PTM dataset, both pLink2 and ECL3 failed to identify any CSMs in the remaining datasets. This outcome highlights the importance of SeaPIC in identifying PTMs within XL-MS datasets.

TABLE 1
Simulated datasets

| | | Simulated datasets (each contains 20,000 spectra) | | | | |
|---|---|---|---|---|---|---|
| | | 0-PTM | 1-PTM | 2-PTM | 3-PTM | 4-PTM |
| Identified | pLink2 | 19916 | 51 | 113 | 52 | 20 |
| CSMs number | ECL3 | 19556 | 44 | 30 | 21 | 18 |

Regarding Table 1, simulated were 100,000 spectra, divided into five datasets, each containing 0, 1, 2, 3, and 4 PTMs. Both pLink22 and ECL33 were employed to analyze the datasets with default settings, but they failed to identify any results in the remaining datasets except for the 0-PTM dataset. In both pLink2 and ECL3, the identified CSMs in the 1-PTM, 2-PTM, 3-PTM, and 4-PTM datasets are incorrectly matched CSMs.

### Synthetic Datasets

Two sets of cross-linked peptides of bovine serum albumin (BSA) protein were prepared using cleavable cross-linker CBDPS13. To incorporate PTMs, the peptides were labeled with light (28.0313 Da) and heavy (34.0631 Da) isotope-coded dimethyl chemicals during the sample preparation. Each set of peptides was subjected to a specific acquisition protocol where one set underwent the CID-MS2-ETD-MS2 acquisition protocol while the other set underwent the HCD-MS2-ETD-MS2 acquisition protocol.

The two synthetic datasets were analyzed using SeaPIC, without specifying the mass of the light and heavy dimethyl. The specific parameters used are included in supplemental Table 2.

TABLE 2
SeaPIC parameters used in the synthetic and real datasets

| | SeaPIC |
|---|---|
| Enzyme | Trypsin |
| Miss_cleavages | 2 |
| Min_length | 5 |
| Known Modifications | C + 57.02 Da |
| | (fixed) |
| | M + 15.99 Da |
| | (variable) |
| MS2 tolerance | 0.01 Da |
| Linker info | CBDPS mₓₗ = 509.097 Da |
| | BS2G mₓₗ = 96.021 Da |
| | BS3 mₓₗ = 138.068 Da |
| Link site | K |

The results obtained from SeaPIC are presented at FIGS. 11 and 12.

Regarding FIGS. 11 and 12, SeaPIC was utilized to analyze two BSA datasets with known ground truth: CID-MS2-ETD-MS2 and HCD-MS2-ETD-MS2. Bar plots were generated to show the results obtained from SeaPIC, displaying the identified PTM masses along with their corresponding scores. Graphs 1100A and 1000B represent the CID and ETD spectra results, respectively, from the CIDMS2-ETD-MS2 dataset, while graphs 1200A and 1200B represent the HCD and ETD spectra results, respectively, from the HCD-MS2-ETD-MS2 dataset.

SeaPIC consistently identified the light and heavy dimethyl masses (marked in red circles) in all scenarios, and these identifications were associated with the highest PTM scores. Additionally, the reported z-scores 185 confirmed the significance of these results.

SeaPIC utilizes b/y ions in the CID and HCD spectra, as well as c/z ions in the ETD spectra, to perform the search. The overall results from SeaPIC demonstrate that it is consistently successful at identifying of the mass of light and heavy dimethyl. These results exhibit the highest PTM scores 184 and significant confidence. The synthetic experiment shows that SeaPIC is capable of fulfilling the task in multiple scenarios, encompassing different spectrum types.

To further compare the effects, pLink2 and ECL3 were run on these synthetic datasets, both with and without utilizing the PTM information provided by SeaPIC. The parameters used in the search engines are detailed in supplemental Table 3.

TABLE 3
Search engine parameters used in synthetic dataset

| Synthetic dataset | pLink2 | ECL3 |
|---|---|---|
| Enzyme | Trypsin | Trypsin |
| Miss_cleavages | 2 | 2 |
| Min_length | 5 | 5 |
| Modifications | (fixed) | (fixed) |
| | C + 57.02 Da | C + 57.02 Da |
| | (variable) | (variable) |
| | M + 15.99 Da | M + 15.99 Da |
| | n-term, K + 28.03 Da | n-term, K + 28.03 Da |
| | n-term, K + 34.06 Da | n-term, K + 34.06 Da |
| MS1 tolerance | 10 ppm | 10 ppm |
| MS2 tolerance | 20 ppm | 20 ppm |
| Linker info | CBDPS mₓₗ = 509.097 Da | |
| Link site | K | K |
| FDR | 0.01 | 0.01 |

The number of identified CSMs is summarized in Table 4:

TABLE 4

| | pLink2 | ECL3 |
|---|---|---|
| | (CSMs number) | (CSMs number) |
| Without SeaPIC | 7 | 0 |
| Using SeaPIC | 136 | 168 |

Regarding Table 4, CSMs numbers of pLink2 and ECL3 using/without using SeaPIC on the synthetic dataset. Observed were a significant difference in results when SeaPIC information was input into these two existing XL-MS search engines. This discrepancy can be attributed, in part, to the efficient labeling method of SeaPIC that leads to the presence of PTMs in almost all cross-linked peptides. Consequently, without prior knowledge of the PTMs in the dataset, the existing XL-MS search engines struggle to identify any matches.

It is evident that without the PTM information from SeaPIC, pLink2 and ECL3 yield minimal identifications. However, when provided with the PTM information from SeaPIC, both search engines successfully identified the labeled cross-linked BSA peptides. This contrast can be attributed to the efficiency of the labeling methods, resulting in the majority of cross-linked peptides carrying mass modifications. Consequently, in the absence of prior knowledge of these modifications, existing XL-MS search engines fail to identify any matches.

### Real Datasets

To investigate the results of SeaPIC on real experimental datasets, three human datasets were employed: PXD04258428 (BS3 cross-linker), PXD04544629 (BS3 cross-linker), and PXD02359330 (BS2G crosslinker).

Generally, these datasets were initially analyzed using SeaPIC. The specific parameters used are included in the supplemental Table 1, provided above. The PTM information, provided by SeaPIC, was incorporated into the search engines pLink2 and ECL3 as variable modifications. The output CSMs from these search engines were filtered to retain the highly confident ones. Finally, a Venn diagram was generated to compare the PTM's consistency from two search engines.

Graphs and Venn diagrams of FIGS. 13 to 15 illustrate the detailed results of the experiments conducted on the three datasets. Graph 1300A, graph 1300B and Venn diagram 1300C correspond to the human dataset PXD04258428 (BS3 cross-linker). Graph 1400A, graph 1400B and Venn diagram 1400C correspond to the human dataset PXD04544629 (BS3 cross-linker). Graph 1500A, graph 1500B and Venn diagram 1500C correspond to the human dataset PXD02359330 (BS2G crosslinker).

More particularly regarding FIGS. 13 to 15, the respective datasets each underwent the same processing pipeline. Initially, SeaPIC was used to screen the data and generate results on a bar plot. Bold dots at graphs 1300A, 1400A and 1500A indicate enriched PTMs or chemical derivatives from a specific cross-linker, that can be verified by the data source. These PTM masses or chemical modifications were either provided by the data source or can be derived from cross-linkers (mono-link mass). Hence, these are considered as true results. All these masses showed high PTM scores, indicating the accuracy of SeaPIC. Indeed, these masses showed high PTM scores.

Subsequently, pLink2 and ECL3 were used with the PTM information from SeaPIC. An FDR value of 0.01 was set, and the identified CSMs had to be identified at least twice for output. The CSMs were then classified as regular or PTM, with the term regular referring to results from the original setting and the term PTM indicating results from the SeaPIC setting (bar graphs 1300B, 1400B and 1500B). The significant number of PTM CSMs (12.4%) suggests that these matches are not random.

Put another way, results were categorized into regular CSMs (identified using original settings) and PTM CSMs (identified using SeaPIC-provided information). It was observed that PTM CSMs represented a significant proportion (12.4%) in both pLink2 and ECL3. The FDR threshold, redundant results requirement, and proportion suggest that the presence of PTM-containing CSMs is unlikely to be a random match.

Put still another way, the confident PTM information obtained from SeaPIC was employed as variable modifications in pLink2 and ECL3. The data was then searched, with an FDR threshold of 0.01, and filtered by keeping only redundant CSMs, identified at least twice. The specific searching parameters used for PXD04258428, PXD045446, and PXD023593 are included in Tables 5 to 7, below.

TABLE 5
Search engine parameters used in the PXD04258428 dataset

| PXD042584 | pLink2 | ECL3 |
|---|---|---|
| Enzyme | Trypsin | Trypsin |
| Miss_cleavages | 2 | 2 |
| Min_length | 5 | 5 |
| Modifications | (fixed) | (fixed) |
| | C + 57.02 Da | C + 57.02 Da |
| | (variable) | (variable) |
| | M + 15.99 Da | M + 15.99 Da |
| | STY + 79.97 Da | STY + 79.97 Da |
| | K + 156.08 Da | K + 156.08 Da |
| MS1 tolerance | 10 ppm | 10 ppm |
| MS2 tolerance | 0.02 Da | 0.02 Da |

| | |
|---|---|
| Linker info | BS3 mₓₗ = 138.068 Da |

| | | |
|---|---|---|
| Link site | K | K |
| FDR | 0.01 | 0.01 |

TABLE 6
Search engine parameters used in the PXD045446dataset

| PXD045446 | pLink2 | ECL3 |
|---|---|---|
| Enzyme | Trypsin | Trypsin |
| Miss_cleavages | 2 | 2 |
| Min_length | 5 | 5 |
| Modifications | (fixed) | (fixed) |
| | C + 57.02 Da | C + 57.02 Da |
| | (variable) | (variable) |
| | M + 15.99 Da | M + 15.99 Da |
| | L + 15.01 Da | L + 15.01 Da |
| | K + 156.08 Da | K + 156.08 Da |
| MS1 tolerance | 10 ppm | 10 ppm |
| MS2 tolerance | 0.02 Da | 0.02 Da |

| | |
|---|---|
| Linker info | BS3 mₓₗ = 138.068 Da |

| | | |
|---|---|---|
| Link site | K | K |
| FDR | 0.01 | 0.01 |

TABLE 7
Search engine parameters used in the PXD023593 dataset

| PXD023593 | pLink2 | ECL3 |
|---|---|---|
| Enzyme | Trypsin | Trypsin |
| Miss_cleavages | 2 | 2 |
| Min_length | 5 | 5 |
| Modifications | (fixed) | (fixed) |
| | C + 57.02 Da | C + 57.02 Da |
| | (variable) | (variable) |
| | M + 15.99 Da | M + 15.99 Da |
| | S + 41.03 Da | S + 41.03 Da |
| | K + 114.03 Da | K + 114.03 Da |
| MS1 tolerance | 10 ppm | 10 ppm |
| MS2 tolerance | 0.02 Da | 0.02 Da |

| | |
|---|---|
| Linker info | BS2G mₓₗ = 96.021 Da |

| | | |
|---|---|---|
| Link site | K | K |
| FDR | 0.01 | 0.01 |

Finally, a Venn diagram using PTM CSMs was generated for each dataset (Venn diagrams 1300C, 1400C and 1500C). It shows substantial overlap between pLink2 and ECL3, confirming the reliability of the PTM information from SeaPIC.

Put another way, these PTM CSMs obtained from both pLink2 and ECL3 were examined and visualized the results in a Venn diagram. The overlap area observed further verifies the validity of the PTM information provided by SeaPIC.

### Linear Peptide Datasets

Due to the limited availability of PTM-containing cross-linked peptides with known ground truth, an alternative approach was used to validate SeaPIC. SeaPIC code was modified to accommodate linear peptide PTM identification, disabling the cross-linking reaction site settings in SeaPIC. This adjustment allowed for exclusively searching for linear peptides with potential PTMs. Furthermore, access to numerous publicly available datasets of PTM-containing linear peptides with known ground truth was employed. Tests were conducted on 21 different PTMs in linear peptides31 (PXD009449) using SeaPIC. All PTMs were identified with the highest PTM scores.

In this regard, these different experiments demonstrate the reliability of SeaPIC and showcase its applicability in scenarios involving linear peptides. A linear peptide searching mode in SeaPIC can also benefit existing XL-MS datasets because in cleavable cross-linking tasks, MS3 spectra are frequently used to generate the linear peptide data.

Discussion turns next to description relative to the SeaPIC workflow provided at FIGS. 2, 3, 4 and 8 in combination.

That is, in a query MS2 spectrum (e.g., XL-MS data 152), SeaPIC (e.g., the generating component 112 of the PTM identifying system 102) can number every peak 204 by their order and pairs every two in a tuple to check if their mass difference 206 is equal to the mass of a known amino acid 147. If so, they can form a tag1 (e.g., tag 172), such as 23-K-25 and 37-F-38 illustrated at FIG. 2. Then SeaPIC can use all the tag1 information to construct a tag graph (e.g., sequence tag graph 170), where each vertex 212 represents the peak number and each edge 216 represents the corresponding amino acid 147. Next, a depth-first search (DFS) algorithm 250 can be adopted to find all the paths 214 in the tag graph (e.g., sequence tag graph 170), sorting them by path length and/or average weighted peak intensity.

Subsequently, the resulting protein sequence can be theoretically digested, and all the peptides can be concatenated using the “$” sign. The peptide database (e.g., information datastore 190) and tags 172 can be encoded into binary form (DB encoding 304 and tag encoding 302), and a bitap (shift-or) algorithm can be utilized for string matching 176, as illustrated at FIG. 3. The matched peptides 322 can be filtered based on the tag position and score, as illustrated at FIG. 4. Then, a fast PTM search method 430 is applied to identify possible PTMs 180 on each peptide, finding the best matches (e.g., identifications 182) using a regularized scoring function Equation 1 (score regularization 460). A PTM score 184 can be assigned based on the score summation from all the MS2 spectra that identified this PTM 180. The results can be reported with a z-score 185 significance criterion (e.g., probability of PTM 180 being comprised by the real peptide set 148; result normalization 800). Finally, the SeaPIC's result (e.g., PTM data 192) can be exported (export of PTM information 900) to an existing XL-MS search engine 194 to find an exact PTM match on the cross-linked peptide.

As further description provided relative to FIGS. 1-15, and particularly of the non-limiting system 100, turning now to FIGS. 16 to 19, an example process flow 1600 can comprise a set of operations for identifying and/or scoring a PTM 180. One or more elements, objects and/or components referenced in the process flow 1600 can be those of FIGS. 1-15. Repetitive description of like elements and/or processes employed in previously described embodiments is omitted for sake of brevity.

At operation 1602, the process flow 1600 can comprise obtaining, by a system (e.g., PTM identifying system 102) comprising at least one processor (e.g., processor 106) and at least one memory (e.g., memory 104) (e.g., obtaining component 110), XL-MS data (e.g., XL-MS data 152) from a mass spectrometry device.

At operation 1604, the process flow 1600 can comprise de-charging, by the system (e.g., generating component 112), the XL-MS data into an alternate file format defining at least precursor mass, charge and mass/charge abundance pairs.

In one or more cases, the XL-MS data 152 can correspond to a real peptide set 148, in a biological system 146, cross-linked by a non-cleavable cross-linking reagent or a cleavable cross-linking reagent 149.

At operation 1606, the process flow 1600 can comprise identifying, by the system (e.g., generating component 112), mass differences between pairs of peaks, of the XL-MS data along the mass/charge (m/z) axis, as corresponding to masses of known amino acids.

At operation 1608, the process flow 1600 can comprise determining, by the system (e.g., generating component 112), whether all pairs of all peaks of the XL-MS data have been identified. If not, the process flow 1600 can proceed back to operation 1606 for continued identifying. If yes, the process flow 1600 can proceed forward to operation 1610.

At operation 1610, the process flow 1600 can comprise generating, by the system (e.g., generating component 112), tags (e.g., tags 172) having respective lengths of one amino acid (e.g., amino acid 147) and a peptide sequence tag graph (e.g., peptide sequence tag graph 170) comprising the tags.

At operation 1612, the process flow 1600 can comprise generating, by the system (e.g., generating component 112), a peptide sequence tag graph (e.g., peptide sequence tag graph 170) based on a dataset of the cross-linking spectral (XL-MS) data defining a real peptide set (e.g., real peptide set 148) of one or more peptides and based on a peptide database (e.g., library datastore 190) comprising information defining known peptides.

At operation 1614, the process flow 1600 can comprise generating, by the system (e.g., generating component 112), the peptide sequence tag graph comprising a system of nodes (e.g., nodes 211) comprising vertices (e.g., vertices 212) and edges (e.g., edges 216), wherein the vertices correspond to peak intensities of respective amino acids (e.g., comprised by the peptides of the real peptide set 148), and wherein the edges represent the respective amino acids corresponding to the peak intensities.

At operation 1616, the process flow 1600 can comprise exploring, by the system (e.g., searching component 114), possible paths (e.g., paths 214) from starting nodes to end nodes, of the system of nodes of the peptide sequence tag graph.

At operation 1618, the process flow 1600 can comprise ranking, by the system (e.g., matching component 116) each path based on a combination of length and sum of weighted intensity thereof.

At operation 1620, the process flow 1600 can comprise employing, by the system (e.g., matching component 116), top ranking paths, from the ranking, and that each comprise at least four amino acids, as input to a fuzzy string matching process (e.g., fuzzy string matching process 176).

At operation 1622, the process flow 1600 can comprise performing, by the system (e.g., matching component 116), the fuzzy string matching process comprising allowing at least one amino acid mismatch between the top ranking paths and peptides of a peptide database, employed for matching to the top ranking paths, resulting in matches (e.g., match 322) corresponding to the candidate peptide set (e.g., candidate peptide set 178).

At operation 1624, the process flow 1600 can comprise, based on the fuzzy string matching process applied to the peptide sequence tag graph, identifying, by the system (e.g., matching component 116), the candidate peptide set corresponding to the real peptide set.

At operation 1626, the process flow 1600 can comprise filtering, by the system (e.g., filtering component 120), the candidate peptide set based on a first threshold (e.g., first threshold 186) equal to a difference between a precursor mass and a residual mass of a cross-linker (e.g., cross-linking reagent 149) corresponding to respective candidate peptides of the candidate peptide set.

At operation 1628, the process flow 1600 can comprise identifying, by the system (e.g., filtering component 120), first respective candidate peptides of the candidate peptide set that satisfy a second threshold (e.g., second threshold 188) that is based on tag positions of the amino acids of the first respective candidate peptides.

At operation 1630, the process flow 1600 can comprise identifying, by the system (e.g., identifying component 126), a post-translational modification (PTM) (e.g., PTM 180) within the candidate peptide set.

At operation 1632, the process flow 1600 can comprise scoring, by the system (e.g., normalizing component 128), the PTM based on an aggregation of additional identifications of the PTM.

At operation 1634, the process flow 1600 can comprise following, by the system (e.g., normalizing component 128), a log-normal distribution and evaluating a z-score for a PTM score (e.g., PTM score 184).

At operation 1636, the process flow 1600 can comprise employing, by the system (e.g., normalizing component 128), a normalizing scoring function (e.g., normalizing scoring function 802) that classifies the PTM, and additional PTMs from additional identifications, based on z-scores (e.g., z-scores 185) of the PTMs, employing an absolute value of a lowest z-score.

At operation 1638, the process flow 1600 can comprise outputting, by the system (e.g., outputting component 130), from the scoring, the PTM score assigned to the PTM that defines a probability of the PTM being comprised by the real peptide set.

At operation 1640, the process flow 1600 can comprise exporting, by the system (e.g., outputting component 130), PTM data (e.g., PTM data 192) comprising the PTM to an XL-MS search engine (e.g., XL-MS search engine 194).

At operation 1642, the process flow 1600 can comprise employing, by the system (e.g., outputting component 130), the PTM data comprising the PTM as a screened input to defining a biological process (e.g., biological process 196) corresponding to the real peptide set based on the identifying of the PTM.

At operation 1644, the process flow 1600 can comprise defining, by the system (e.g., outputting component 130), the biological process corresponding to the real peptide set based on the identifying of the PTM.

### Context for Example Embodiments

For simplicity of explanation, the computer-implemented methodologies and/or processes provided herein are depicted and/or described as a series of acts. The subject application is not limited by the acts illustrated and/or by the order of acts, for example acts can occur in one or more orders and/or concurrently, and with other acts not presented and described herein. The operations of process flows of the figures provided herein are example operations, and there can be one or more embodiments that implement more or fewer operations than are depicted.

Furthermore, not all illustrated acts can be utilized to implement the computer-implemented methodologies in accordance with the described subject matter. In addition, the computer-implemented methodologies could alternatively be represented as a series of interrelated states via a state diagram or events. Additionally, the computer-implemented methodologies described hereinafter and throughout this specification are capable of being stored on an article of manufacture to facilitate transporting and transferring the computer-implemented methodologies to computers. The term article of manufacture, as used herein, is intended to encompass a computer program accessible from any machine-readable device or storage media.

In this regard, one or more systems, computer readable mediums and/or computer-implemented methods provided herein and/or described herein relate to identifying post-translational modifications in cross-linking mass spectrometry data. An example system can comprise at least one processor 106, and at least one memory 104 that stores executable instructions that, when executed by the at least one processor 106, facilitates performance of operations, comprising generating a peptide sequence tag graph 170 based on a dataset of cross-linking spectral (XL-MS) data 152 defining a real peptide set 148 of one or more peptides and based on a peptide database 190 comprising information defining known peptides, based on a fuzzy string matching process 176 applied to the peptide sequence tag graph 170, identifying a candidate peptide set 178 corresponding to the real peptide set 148, identifying a post-translational modification (PTM) 180 within the candidate peptide set 178, and scoring the PTM 180 based on an aggregation of additional identifications 182 of the PTM 180, wherein the scoring results in a PTM score 184 assigned to the PTM 180 that defines a probability of the PTM 180 being comprised by the real peptide set 148.

The one or more example embodiments described herein can be implemented within, in connection with and/or coupled to an imaging device, imaging system, scientific measurement device, and/or scientific measurement system, such as the MS device 150.

Indeed, in view of the one or more example embodiments described herein, a practical application of the one or more systems, computer-implemented methods and/or computer readable mediums described herein can be the versatility of use of the one or more embodiments described herein. That is, the method of searching PTMs in cross-linking mass spectrometry data (SeaPIC) described herein can be employed with different spectrum types, cross-linker (e.g., cross-linking reagent) types and/or task types (e.g., cross-linked peptides vs. linear peptides). For example, SeaPIC can be employed with various types of spectra, including, but not limited to, collision-induced dissociation (CID) spectra, high-energy collisional dissociation (HCD) spectra, and electron-transfer dissociation (ETD) spectra, and is capable of addressing both non-cleavable and cleavable cross-linking scenarios. These are useful and practical applications of computers, thus providing enhanced (e.g., improved and/or optimized) analytical model training, sample preparation and/or imaging system use. Overall, such computerized tools can constitute a concrete and tangible technical improvement in the fields of material analysis, and more particularly in employing scientific measurement systems, such as including, but not limited to, the fields of biology, such as peptide sequencing.

Furthermore, one or more example embodiments described herein can be employed in a real-world system based on the disclosed teachings. For example, the one or more example embodiments described herein can provide an input set of data, comprising probable PTMs and/or peptide matches, to an XL-MS search engine, allowing for a more reliable and/or efficient analysis by the XL-MS search engine. That is, the XL-MS technique is a data-heavy and time-intensive technique that can be sped up using a combination of the one or more embodiments described herein and an existing XL-MS search engine using output of the one or more embodiments described herein. Put another way, the screening capabilities provided by the one or more embodiments described herein can extract PTM information from data, and enhance the performance of existing XL-MS search engines. Indeed, using the one or more embodiments described herein, PTM information can be identified that is otherwise not identified using only an existing XL-MS search engine. The embodiments disclosed herein thus can provide improvements to scientific instrument technology (e.g., improvements in the computer technology supporting such scientific instruments, among other improvements).

The systems and/or devices have been (and/or will be further) described herein with respect to interaction between one or more components. Such systems and/or components can include those components or sub-components specified therein, one or more of the specified components and/or sub-components, and/or additional components. Sub-components can be implemented as components communicatively coupled to other components rather than included within parent components. One or more components and/or sub-components can be combined into a single component providing aggregate functionality. The components can interact with one or more other components not specifically described herein for the sake of brevity, but known by those of skill in the art.

One or more example embodiments described herein can be, in one or more example embodiments, inherently and/or inextricably tied to computer technology and cannot be implemented outside of a computing environment. For example, one or more processes performed by one or more example embodiments described herein can more efficiently, and even more feasibly, provide program and/or program instruction execution, such as relative to identification and analysis of post-translational modifications, as compared to existing systems and/or techniques. Systems, computer-implemented methods and/or computer readable mediums providing performance of these processes are of great utility in the fields of material analysis and cannot be equally practicably implemented in a sensible way outside of a computing environment.

One or more example embodiments described herein can employ hardware and/or software to solve problems that are highly technical, that are not abstract, and that cannot be performed as a set of mental acts by a human. For example, a human, or even thousands of humans, cannot efficiently, accurately and/or effectively analyze computer data/metadata (e.g., XL-MS data, peptide sequence tag graph data, PTM data and/or candidate peptide data) describing peptide sequences, amino acids, cross-linking reagents and/or the like, as the one or more example embodiments described herein can provide this process. Moreover, neither can the human mind nor a human with pen and paper conduct one or more of these processes, as conducted by one or more example embodiments described herein.

In one or more example embodiments, one or more of the processes described herein can be performed by one or more specialized computers (e.g., a specialized processing unit, a specialized classical computer, and/or another type of specialized computer) to execute defined tasks related to the one or more technologies describe above. One or more example embodiments described herein and/or components thereof can be employed to solve new problems that arise through advancements in technologies mentioned above, employment of cloud computing systems, computer architecture and/or another technology.

One or more example embodiments described herein can be fully operational towards performing one or more other functions (e.g., fully powered on, fully executed and/or another function) while also performing one or more of the one or more operations described herein.

The paragraphs that follow provide additional summaries describing example devices, systems, methods and/or non-transitory mediums.

An example system can comprise at least one processor and at least one memory that stores executable instructions that, when executed by the at least one processor, facilitates performance of operations. The operations can comprise generating a peptide sequence tag graph based on a dataset of cross-linking spectral (XL-MS) data defining a real peptide set of one or more peptides and based on a peptide database comprising information defining known peptides, based on a fuzzy string matching process applied to the peptide sequence tag graph, identifying a candidate peptide set corresponding to the real peptide set, identifying a post-translational modification (PTM) within the candidate peptide set, and scoring the PTM based on an aggregation of additional identifications of the PTM. The scoring results in a PTM score assigned to the PTM that defines a probability of the PTM being comprised by the real peptide set.

The system of any preceding paragraph can comprise the processor facilitating operations further comprising identifying mass differences between pairs of peaks, of the XL-MS data along the mass/charge (m/z) axis, as corresponding to masses of known amino acids, generating tags having respective lengths of one amino acid, and generating the peptide sequence tag graph comprising the tags.

The system of any preceding paragraph can comprise the processor facilitating operations further comprising generating the peptide sequence tag graph comprising a system of nodes comprising vertices and edges, wherein the vertices correspond to peak intensities of the respective amino acids, and wherein the edges represent the respective amino acids corresponding to the peak intensities.

The system of any preceding paragraph can comprise the processor facilitating operations further comprising exploring possible paths from starting nodes to end nodes, of a system of nodes of the peptide sequence tag graph, and ranking each path based on a combination of length and sum of weighted intensity thereof, and employing top ranking paths, from the ranking, and that each comprise at least four amino acids, as input to the fuzzy string matching process.

The system of any preceding paragraph can comprise the processor facilitating operations further comprising performing the fuzzy string matching process comprising allowing at least one amino acid mismatch between the top ranking paths and peptides of a peptide database, employed for matching to the top ranking paths, resulting in matches corresponding to the candidate peptide set.

The system of any preceding paragraph can comprise the processor facilitating operations further comprising, prior to the scoring, filtering the candidate peptide set based on a first threshold equal to a difference between a precursor mass and a residual mass of a cross-linking reagent corresponding to respective candidate peptides of the candidate peptide set, and identifying first respective candidate peptides of the candidate peptide set that satisfy a second threshold that is based on tag positions of the amino acids of the first respective candidate peptides.

The system of any preceding paragraph can comprise wherein the scoring the PTM comprises following a log-normal distribution and evaluating a z-score for the PTM score.

The system of any preceding paragraph can comprise the processor facilitating operations further comprising obtaining the XL-MS data from a mass spectrometry device, wherein the XL-MS data corresponds to the real peptide set, in a biological system, cross-linked by a non-cleavable cross-linking reagent or a cleavable cross-linking reagent.

The system of any preceding paragraph can comprise the processor facilitating operations further comprising, obtaining the XL-MS data from a mass spectrometry device, and de-charging the XL-MS data into an alternate file format defining at least precursor mass, charge and mass/charge abundance pairs.

The system of any preceding paragraph can comprise the processor facilitating operations further comprising, exporting PTM data comprising the PTM to an XL-MS search engine.

The system of any preceding paragraph can comprise the processor facilitating operations further comprising, defining a biological process corresponding to the real peptide set based on the identifying of the PTM.

An example method can comprise generating, by a system comprising at least one processor, a peptide sequence tag graph based on a dataset of cross-linking spectral (XL-MS) data defining a real peptide set of one or more peptides and a peptides database comprising information defining peptides, the generating comprising: identifying mass differences between pairs of peaks, of the XL-MS data along the mass/charge (m/z) axis, as corresponding to masses of known amino acids, generating tags having respective lengths of one amino acid, and generating the peptide sequence tag graph comprising the tags, identifying a candidate peptide set from the peptide sequence tag graph and corresponding to the real peptide set, identifying a post-translational modification (PTM) within the candidate peptide set, and scoring the PTM resulting in a PTM score assigned to the PTM that defines a probability of the PTM being comprised by the real peptide set.

The method of any preceding paragraph can further comprise generating the peptide sequence tag graph having a system of nodes comprising vertices and edges, wherein the vertices correspond to peak intensities of the respective amino acids, and wherein the edges represent the respective amino acids corresponding to the peak intensities.

The method of any preceding paragraph can further comprise exploring possible paths from starting nodes to end nodes of a system of nodes of the peptide sequence tag graph, and ranking each path based on a combination of length and sum of weighted intensity thereof.

The method of any preceding paragraph can further comprise employing top ranking paths, from the ranking, and that each comprise at least four amino acids, as input to the identifying the candidate peptide set.

The method of any preceding paragraph can further comprise wherein the scoring the PTM comprises: employing a normalizing scoring function that classifies the PTM, and additional PTMs from additional identifications, based on z-scores of the PTMs, employing an absolute value of a lowest z-score.

The method of any preceding paragraph can further comprise employing the PTM data comprising the PTM as a screened input to defining a biological process corresponding to the real peptide set based on the identifying of the PTM.

An example non-transitory machine-readable medium can comprise executable instructions that, when executed by at least one processor facilitate performance of operations, comprising generating a peptide sequence tag graph based on a dataset of cross-linking spectral (XL-MS) data defining a real peptide set of one or more peptides and a protein database comprising information defining proteins, evaluating amino acid tags of the peptide sequence tag graph, comprising: exploring possible paths from starting nodes to end nodes of a system of nodes of the peptide sequence tag graph, ranking each path based on a combination of length and sum of weighted intensity thereof, and employing top ranking paths, from the ranking, and that each comprise at least four amino acids, as input to a fuzzy string matching process, based on the fuzzy string matching process, identifying a candidate peptide set corresponding to the real peptide set, identifying a post-translational modification (PTM) within the candidate peptide set, and scoring the PTM based on an aggregation of additional identifications of the PTM, wherein the scoring results in a PTM score assigned to the PTM that defines a probability of the PTM being comprised by the real peptide set.

The non-transitory machine-readable medium of any preceding paragraph can comprise executable instructions that, when executed, facilitate performance of operations further comprising performing the fuzzy string matching process comprising allowing at least one amino acid mismatch between the top ranking paths and peptides of a peptide database, employed for matching to the top ranking paths, resulting in matches corresponding to the candidate peptide set.

The non-transitory machine-readable medium of any preceding paragraph can comprise executable instructions that, when executed, facilitate performance of operations further comprising employing the PTM data comprising the PTM as a screened input to an XL-MS search engine, and using the XL-MS search engine, defining a biological process corresponding to the real peptide set based on the identifying of the PTM.

An identification method for identifying post-translational modifications (PTMs) in cross-linking mass spectrometry (XL-MS) data can comprise using a cross-linking dataset (applicable to MS2 spectra only) and the corresponding protein database as the input, de-charging the MS2 data into MGF format, extracting tag information, constructing tag graph and pruning the tag graph, using fuzzy string matching (bitap) algorithm to find the peptide candidates, matching the PTMs by the regularized scoring function and using a greedy approach to accelerating the PTM matching process, and normalizing the result and exporting the PTM information to the existing XL-MS search engine.

The identification method of any preceding paragraph can comprise wherein the cross-linked data are obtained from proteins in a biological system cross-linked by a non-cleavable or cleavable crosslinking reagent.

The identification method of any preceding paragraph can further comprise filtering, enriching, and digesting the cross-linked proteins to produce the cross-linked peptides.

The identification method of any preceding paragraph can comprise wherein the biological system includes cells, tissues, blood, serum, sputum, etc.

The identification method of any preceding paragraph can comprise wherein the samples are digested into peptides by an enzyme before mass spectrometry data acquisition.

The identification method of any preceding paragraph can comprise wherein after the digestion, the cross-linked samples are sent to tandem mass spectrometry.

The identification method of any preceding paragraph can comprise wherein the de-charging uses Mascot Distiller as the de-charging tool.

The identification method of any preceding paragraph can comprise wherein using fuzzy string matching comprises one mismatch setting for the fuzzy string match algorithm without considering any string deletion or insertion.

The identification method of any preceding paragraph can comprise wherein matching the PTMs comprises using a regularized Xcorr scoring function that is designed specifically for PTM matching and a fast-searching method that sequentially determines PTMs.

The identification method of any preceding paragraph can comprise wherein exporting the PTM information comprises exporting the normalized PTM information to an existing XL-MS search engines to match the PTM-containing cross-linked peptides finally.

The identification method of any preceding paragraph can comprise wherein the exported PTM information is treated as variable modifications as the input for XL-MS search engines.

### Example Operating Environment

FIG. 20 is a schematic block diagram of an operating environment 2000 with which the described subject matter can interact. The operating environment 2000 comprises one or more remote component(s) 2010. The remote component(s) 2010 can be hardware and/or software (e.g., threads, processes, computing devices). In one or more embodiments, remote component(s) 2010 can be a distributed computer system, connected to a local automatic scaling component and/or programs that use the resources of a distributed computer system, via communication framework 2040. Communication framework 2040 can comprise wired network devices, wireless network devices, mobile devices, wearable devices, radio access network devices, gateway devices, femtocell devices, servers, etc.

The operating environment 2000 also comprises one or more local component(s) 2020. The local component(s) 2020 can be hardware and/or software (e.g., threads, processes, computing devices). In one or more embodiments, local component(s) 2020 can comprise an automatic scaling component and/or programs that communicate/use the remote resources 2010 and 2020, etc., connected to a remotely located distributed computing system via communication framework 2040.

One possible communication between a remote component(s) 2010 and a local component(s) 2020 can be in the form of a data packet adapted to be transmitted between two or more computer processes. Another possible communication between a remote component(s) 2010 and a local component(s) 2020 can be in the form of circuit-switched data adapted to be transmitted between two or more computer processes in radio time slots. The operating environment 2000 comprises a communication framework 2040 that can be employed to facilitate communications between the remote component(s) 2010 and the local component(s) 2020, and can comprise an air interface, e.g., interface of a UMTS network, via an LTE network, etc. Remote component(s) 2010 can be operably connected to one or more remote data store(s) 2050, such as a hard drive, solid state drive, subscriber identity module (SIM) card, electronic SIM (eSIM), device memory, etc., that can be employed to store information on the remote component(s) 2010 side of communication framework 2040. Similarly, local component(s) 2020 can be operably connected to one or more local data store(s) 2030, that can be employed to store information on the local component(s) 2020 side of communication framework 2040.

### Example Computing Environment

In order to provide additional context for various embodiments described herein, FIG. 21 and the following discussion are intended to provide a brief, general description of a suitable computing environment 2100 in which the various embodiments of the embodiment described herein can be implemented. While the embodiments have been described above in the general context of computer-executable instructions that can run on one or more computers, those skilled in the art will recognize that the embodiments can also be implemented in combination with other program modules and/or as a combination of hardware and software.

Generally, program modules include routines, programs, components, data structures, etc., that perform tasks or implement abstract data types. Moreover, the methods can be practiced with other computer system configurations, including single-processor or multiprocessor computer systems, minicomputers, mainframe computers, Internet of Things (IoT) devices, distributed computing systems, as well as personal computers, hand-held computing devices, microprocessor-based or programmable consumer electronics, and the like, each of which can be operatively coupled to one or more associated devices.

The illustrated embodiments of the embodiments herein can also be practiced in distributed computing environments where certain tasks are performed by remote processing devices that are linked through a communications network. In a distributed computing environment, program modules can be located in both local and remote memory storage devices.

Computing devices typically include a variety of media, which can include computer-readable storage media, machine-readable storage media, and/or communications media, which two terms are used herein differently from one another as follows. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data, or unstructured data.

Computer-readable storage media can include, but are not limited to, random access memory (RAM), read only memory (ROM), electrically erasable programmable read only memory (EEPROM), flash memory or other memory technology, compact disk read only memory (CD-ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible and/or non-transitory media which can be used to store desired information. In this regard, the terms “tangible” or “non-transitory” herein as applied to storage, memory, or computer-readable media, exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries, or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and include any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

Referring still to FIG. 21, the example computing environment 2100 which can implement one or more embodiments described herein includes a computer 2102, the computer 2102 including a processing unit 2104, a system memory 2106 and a system bus 2108. The system bus 1008 couples system components including, but not limited to, the system memory 2106 to the processing unit 2104. The processing unit 2104 can be any of various commercially available processors. Dual microprocessors and other multi-processor architectures can also be employed as the processing unit 2104.

The system bus 2108 can be any of several types of bus structure that can further interconnect to a memory bus (with or without a memory controller), a peripheral bus, and a local bus using any of a variety of commercially available bus architectures. The system memory 2106 includes ROM 2110 and RAM 2112. A basic input/output system (BIOS) can be stored in a non-volatile memory such as ROM, erasable programmable read only memory (EPROM), EEPROM, which BIOS contains the basic routines that help to transfer information between elements within the computer 2102, such as during startup. The RAM 2112 can also include a high-speed RAM such as static RAM for caching data.

The computer 2102 further includes an internal hard disk drive (HDD) 2114 (e.g., EIDE, SATA), and can include one or more external storage devices 2116 (e.g., a magnetic floppy disk drive (FDD) 2116, a memory stick or flash drive reader, a memory card reader, etc.). While the internal HDD 2114 is illustrated as located within the computer 2102, the internal HDD 2114 can also be configured for external use in a suitable chassis (not shown). Additionally, while not shown in computing environment 2100, a solid-state drive (SSD) could be used in addition to, or in place of, an HDD 2114.

Other internal or external storage can include at least one other storage device 2120 with storage media 2122 (e.g., a solid-state storage device, a nonvolatile memory device, and/or an optical disk drive that can read or write from removable media such as a CD-ROM disc, a DVD, a BD, etc.). The external storage 2116 can be facilitated by a network virtual machine. The HDD 2114, external storage device 2116 and storage device (e.g., drive) 2120 can be connected to the system bus 2108 by an HDD interface 2124, an external storage interface 2126 and a drive interface 2128, respectively.

The drives and their associated computer-readable storage media provide nonvolatile storage of data, data structures, computer-executable instructions, and so forth. For the computer 2102, the drives and storage media accommodate the storage of any data in a suitable digital format. Although the description of computer-readable storage media above refers to respective types of storage devices, other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 2112, including an operating system 2130, one or more application programs 2132, other program modules 2134 and program data 2136. All or portions of the operating system, applications, modules, and/or data can also be cached in the RAM 2112. The systems and methods described herein can be implemented utilizing various commercially available operating systems or combinations of operating systems.

Computer 2102 can optionally comprise emulation technologies. For example, a hypervisor (not shown) or other intermediary can emulate a hardware environment for operating system 2130, and the emulated hardware can optionally be different from the hardware illustrated in FIG. 21. In such an embodiment, operating system 2130 can comprise one virtual machine (VM) of multiple VMs hosted at computer 2102. Furthermore, operating system 2130 can provide runtime environments, such as the Java runtime environment or the .NET framework, for applications 2132. Runtime environments are consistent execution environments that allow applications 2132 to run on any operating system that includes the runtime environment. Similarly, operating system 2130 can support containers, and applications 2132 can be in the form of containers, which are lightweight, standalone, executable packages of software that include, e.g., code, runtime, system tools, system libraries and settings for an application.

Further, computer 2102 can be enabled with a security module, such as a trusted processing module (TPM). For instance, with a TPM, boot components hash next in time boot components, and wait for a match of results to secured values, before loading a next boot component. This process can take place at any layer in the code execution stack of computer 2102, e.g., applied at the application execution level or at the operating system (OS) kernel level, thereby enabling security at any level of code execution.

A user can enter commands and information into the computer 2102 through one or more wired/wireless input devices, e.g., a keyboard 2138, a touch screen 2140, and a pointing device, such as a mouse 2142. Other input devices (not shown) can include a microphone, an infrared (IR) remote control, a radio frequency (RF) remote control, or other remote control, a joystick, a virtual reality controller and/or virtual reality headset, a game pad, a stylus pen, an image input device, e.g., camera, a gesture sensor input device, a vision movement sensor input device, an emotion or facial detection device, a biometric input device, e.g., fingerprint or iris scanner, or the like. These and other input devices are often connected to the processing unit 2104 through an input device interface 2144 that can be coupled to the system bus 2108, but can be connected by other interfaces, such as a parallel port, an IEEE 1394 serial port, a game port, a USB port, an IR interface, a BLUETOOTH® interface, etc.

A monitor 2146 or other type of display device can also be connected to the system bus 2108 via an interface, such as a video adapter 2148. In addition to the monitor 2146, a computer typically includes other peripheral output devices (not shown), such as speakers, printers, etc.

The computer 2102 can operate in a networked environment using logical connections via wired and/or wireless communications to one or more remote computers, such as a remote computer 2150. The remote computer 2150 can be a workstation, a server computer, a router, a personal computer, portable computer, microprocessor-based entertainment appliance, a peer device or other common network node, and typically includes many or all of the elements described relative to the computer 2102, although, for purposes of brevity, only a memory/storage device 2152 is illustrated. The logical connections depicted include wired/wireless connectivity to a local area network (LAN) 2154 and/or larger networks, e.g., a wide area network (WAN) 2156. Such LAN and WAN networking environments are commonplace in offices and companies, and facilitate enterprise-wide computer networks, such as intranets, all of which can connect to a global communications network, e.g., the Internet.

When used in a LAN networking environment, the computer 2102 can be connected to the local network 2154 through a wired and/or wireless communication network interface or adapter 2158. The adapter 2158 can facilitate wired or wireless communication to the LAN 2154, which can also include a wireless access point (AP) disposed thereon for communicating with the adapter 2158 in a wireless mode.

When used in a WAN networking environment, the computer 2102 can include a modem 2160 or can be connected to a communications server on the WAN 2156 via other means for establishing communications over the WAN 2156, such as by way of the Internet. The modem 2160, which can be internal or external and a wired or wireless device, can be connected to the system bus 2108 via the input device interface 2144. In a networked environment, program modules depicted relative to the computer 2102 or portions thereof, can be stored in the remote memory/storage device 2152. The network connections shown are examples and other means of establishing a communications link between the computers can be used.

When used in either a LAN or WAN networking environment, the computer 2102 can access cloud storage systems or other network-based storage systems in addition to, or in place of, external storage devices 2116 as described above. Generally, a connection between the computer 2102 and a cloud storage system can be established over a LAN 2154 or WAN 2156 e.g., by the adapter 2158 or modem 2160, respectively. Upon connecting the computer 2102 to an associated cloud storage system, the external storage interface 2126 can, with the aid of the adapter 2158 and/or modem 2160, manage storage provided by the cloud storage system as it would other types of external storage. For instance, the external storage interface 2126 can be configured to provide access to cloud storage sources as if those sources were physically connected to the computer 2102.

The computer 2102 can be operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, store shelf, etc.), and telephone. This can include Wireless Fidelity (Wi-Fi) and BLUETOOTH® wireless technologies. Thus, the communication can be a defined structure as with a conventional network or simply an ad hoc communication between at least two devices.

### CONCLUSION

The above description of illustrated embodiments of the one or more embodiments described herein, comprising what is described in the Abstract, is not intended to be exhaustive or to limit the described embodiments to the precise forms described. While one or more specific embodiments and examples are described herein for illustrative purposes, various modifications are possible that are considered within the scope of such embodiments and examples, as those skilled in the relevant art can recognize.

In this regard, while the described subject matter has been described in connection with various embodiments and corresponding figures, where applicable, other similar embodiments can be used or modifications and additions can be made to the described embodiments for performing the same, similar, alternative, or substitute function of the described subject matter without deviating therefrom. Therefore, the described subject matter should not be limited to any single embodiment described herein, but rather should be construed in breadth and scope in accordance with the appended claims below.

As it employed in the subject specification, the term “processor” can refer to substantially any computing processing unit or device comprising, but not limited to comprising, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit, a digital signal processor, a field programmable gate array, a programmable logic controller, a complex programmable logic device, a discrete gate or transistor logic, discrete hardware components, or any combination thereof designed to perform the functions described herein. Processors can exploit nano-scale architectures to optimize space usage or enhance performance of user equipment. A processor can also be implemented as a combination of computing processing units.

As used in this application, the terms “component,” “system,” “platform,” “layer,” “selector,” “interface,” and the like are intended to refer to a computer-related entity or an entity related to an operational apparatus with one or more functionalities, wherein the entity can be either hardware, a combination of hardware and software, software, or software in execution. As an example, a component may be, but is not limited to being, a process running on a processor, a processor, an object, an executable, a thread of execution, a program, and/or a computer. By way of illustration and not limitation, both an application running on a server and the server can be a component. One or more components may reside within a process and/or thread of execution and a component may be localized on one computer and/or distributed between two or more computers. In addition, these components can execute from various computer readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal). As another example, a component can be an apparatus with functionality provided by mechanical parts operated by electric or electronic circuitry, which is operated by a software or a firmware application executed by a processor, wherein the processor can be internal or external to the apparatus and executes at least a part of the software or firmware application. As yet another example, a component can be an apparatus that provides functionality through electronic components without mechanical parts, the electronic components can comprise a processor therein to execute software or firmware that confers at least in part the functionality of the electronic components.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of these instances.

While the embodiments are susceptible to various modifications and alternative constructions, certain illustrated implementations thereof are shown in the drawings and have been described above in detail. However, there is no intention to limit the various embodiments to the one or more specific forms described, but on the contrary, the intention is to cover all modifications, alternative constructions, and equivalents falling within the spirit and scope.

In addition to the various implementations described herein, other similar implementations can be used, or modifications and additions can be made to the described implementation for performing the same or equivalent function of the corresponding implementation without deviating therefrom. Still further, multiple processing chips or multiple devices can share the performance of one or more functions described herein, and similarly, storage can be implemented across different devices. Accordingly, the various embodiments are not to be limited to any single implementation, but rather are to be construed in breadth, spirit, and scope in accordance with the appended claims.

What is claimed is:

1. A system, comprising:
at least one processor; and
at least one memory that stores executable instructions that, when executed by the at least one processor, facilitates performance of operations, comprising:
generating a peptide sequence tag graph based on a dataset of cross-linking spectral (XL-MS) data defining a real peptide set of one or more peptides and based on a peptide database comprising information defining known peptides;
based on a fuzzy string matching process applied to the peptide sequence tag graph, identifying a candidate peptide set corresponding to the real peptide set;
identifying a post-translational modification (PTM) within the candidate peptide set; and
scoring the PTM based on an aggregation of additional identifications of the PTM,
wherein the scoring results in a PTM score assigned to the PTM that defines a probability of the PTM being comprised by the real peptide set.

2. The system of claim 1, wherein the operations further comprise:
identifying mass differences between pairs of peaks, of the XL-MS data along the mass/charge (m/z) axis, as corresponding to masses of known amino acids;
generating tags having respective lengths of one amino acid; and
generating the peptide sequence tag graph comprising the tags.

3. The system of claim 2, wherein the operations further comprise:
generating the peptide sequence tag graph comprising a system of nodes comprising vertices and edges,
wherein the vertices correspond to peak intensities of the respective amino acids, and
wherein the edges represent the respective amino acids corresponding to the peak intensities.

4. The system of claim 1, wherein the operations further comprise:
exploring possible paths from starting nodes to end nodes, of a system of nodes of the peptide sequence tag graph; and
ranking each path based on a combination of length and sum of weighted intensity thereof; and
employing top ranking paths, from the ranking, and that each comprise at least four amino acids, as input to the fuzzy string matching process.

5. The system of claim 4, wherein the operations further comprise:
performing the fuzzy string matching process comprising allowing at least one amino acid mismatch between the top ranking paths and peptides of a peptide database, employed for matching to the top ranking paths, resulting in matches corresponding to the candidate peptide set.

6. The system of claim 1, wherein the operations further comprise:
prior to the scoring, filtering the candidate peptide set based on a first threshold equal to a difference between a precursor mass and a residual mass of a cross-linking reagent corresponding to respective candidate peptides of the candidate peptide set; and
identifying first respective candidate peptides of the candidate peptide set that satisfy a second threshold that is based on tag positions of the amino acids of the first respective candidate peptides.

7. The system of claim 1, wherein the scoring the PTM comprises following a log-normal distribution and evaluating a z-score for the PTM score.

8. The system of claim 1, wherein the operations further comprise:
obtaining the XL-MS data from a mass spectrometry device,
wherein the XL-MS data corresponds to the real peptide set, in a biological system, cross-linked by a non-cleavable cross-linking reagent or a cleavable cross-linking reagent.

9. The system of claim 1, wherein the operations further comprise:
obtaining the XL-MS data from a mass spectrometry device; and
de-charging the XL-MS data into an alternate file format defining at least precursor mass, charge and mass/charge abundance pairs.

10. The system of claim 1, wherein the operations further comprise:
exporting PTM data comprising the PTM to an XL-MS search engine.

11. The system of claim 1, wherein the operations further comprise:
defining a biological process corresponding to the real peptide set based on the identifying of the PTM.

12. A method, comprising:
generating, by a system comprising at least one processor, a peptide sequence tag graph based on a dataset of cross-linking spectral (XL-MS) data defining a real peptide set of one or more peptides and a peptides database comprising information defining peptides, the generating comprising:
identifying mass differences between pairs of peaks, of the XL-MS data along the mass/charge (m/z) axis, as corresponding to masses of known amino acids,
generating tags having respective lengths of one amino acid, and
generating the peptide sequence tag graph comprising the tags;
identifying a candidate peptide set from the peptide sequence tag graph and corresponding to the real peptide set;
identifying a post-translational modification (PTM) within the candidate peptide set; and
scoring the PTM resulting in a PTM score assigned to the PTM that defines a probability of the PTM being comprised by the real peptide set.

13. The method of claim 12, further comprising:
generating the peptide sequence tag graph having a system of nodes comprising vertices and edges,
wherein the vertices correspond to peak intensities of the respective amino acids, and
wherein the edges represent the respective amino acids corresponding to the peak intensities.

14. The method of claim 12, further comprising:
exploring possible paths from starting nodes to end nodes of a system of nodes of the peptide sequence tag graph; and
ranking each path based on a combination of length and sum of weighted intensity thereof.

15. The method of claim 14, further comprising:
employing top ranking paths, from the ranking, and that each comprise at least four amino acids, as input to the identifying the candidate peptide set.

16. The method of claim 12, wherein the scoring the PTM comprises:
employing a normalizing scoring function that classifies the PTM, and additional PTMs from additional identifications, based on z-scores of the PTMs, employing an absolute value of a lowest z-score.

17. The method of claim 12, further comprising:
employing the PTM data comprising the PTM as a screened input to defining a biological process corresponding to the real peptide set based on the identifying of the PTM.

18. A non-transitory machine-readable medium, comprising executable instructions that, when executed by at least one processor facilitate performance of operations, comprising:
generating a peptide sequence tag graph based on a dataset of cross-linking spectral (XL-MS) data defining a real peptide set of one or more peptides and a protein database comprising information defining proteins;
evaluating amino acid tags of the peptide sequence tag graph, comprising:
exploring possible paths from starting nodes to end nodes of a system of nodes of the peptide sequence tag graph,
ranking each path based on a combination of length and sum of weighted intensity thereof, and
employing top ranking paths, from the ranking, and that each comprise at least four amino acids, as input to a fuzzy string matching process;
based on the fuzzy string matching process, identifying a candidate peptide set corresponding to the real peptide set;
identifying a post-translational modification (PTM) within the candidate peptide set; and
scoring the PTM based on an aggregation of additional identifications of the PTM,
wherein the scoring results in a PTM score assigned to the PTM that defines a probability of the PTM being comprised by the real peptide set.

19. The non-transitory machine-readable medium of claim 18, wherein the operations further comprise:
performing the fuzzy string matching process comprising allowing at least one amino acid mismatch between the top ranking paths and peptides of a peptide database, employed for matching to the top ranking paths, resulting in matches corresponding to the candidate peptide set.

20. The non-transitory machine-readable medium of claim 18, wherein the operations further comprise:
employing the PTM data comprising the PTM as a screened input to an XL-MS search engine; and
using the XL-MS search engine, defining a biological process corresponding to the real peptide set based on the identifying of the PTM.
